(19) United States
(12) Patent Application Publication
(10) Pub. No.: US 2011/0035019 A1
(43) Pub. Date: Feb. 10, 2011

(54) TOTAL ANKLE REPLACEMENT SYSTEM

(75) Inventors: Tarun Goswami, Beavercreek, OH (US); Shawn Gargac, Fort Wayne, IN (US); Richard Laughlin, Beavercreek, OH (US); Ashkahn Golshani, Beavercreek, OH (US); Junitha Michael, Rochester, NY (US)

Correspondence Address: DINSMORE & SHOHL LLP, FIFTH THIRD CENTER, ONE SOUTH MAIN STREET, SUITE 1300, DAYTON, OH, 45402-2023, US

(73) Assignee: WRIGHT STATE UNIVERSITY, Dayton, OH (US)

(21) Appl. No.: 12/833,704

(22) Filed: Jul. 9, 2010

Related U.S. Application Data
(60) Provisional application No. 61/224,263, filed on Jul. 9, 2009.

(51) Int. Cl. A61F 2/42

(52) U.S. Cl. 623/21.18

(57) ABSTRACT

A total ankle replacement system is presented. The total ankle joint replacement procedure can be used to treat persons with disability, deformity, or that are suffering from osteoarthritis and other arthritic conditions. The total ankle joint assembly generally comprises a tibial component, a talar component, and a bearing component, where the bearing component is positioned between and articulates with the tibial component and talar component to mimic the natural ankle joint movement.

### RELATED APPLICATIONS

This application claims the benefit of U.S. Provisional Patent Application Ser. No. 61/224,263, filed Jul. 9, 2009, and entitled “Total Ankle Replacement System,” which is incorporated herein by reference.

### BACKGROUND

Injured or diseased ankle joints can occur for a variety of reasons, such as, rheumatoid arthritis, osteoarthritis, a bone fracture or arthritis caused from a prior ankle injury or prior ankle surgery. Some options to correct failed ankle joints can include ankle fusion where the joint is fused or glued together, which can limit the up and down movement, and total ankle arthroplasty where the ankle joint is removed and replaced with an artificial substitute.

An ankle replacement can allow for fairly normal ankle movement. In addition, ankle replacement surgery may be preferred to an ankle fusion procedure as it may prevent the stress that can accumulate in joints next to the ankle when the ankle joint is fused together. The fused joint can cause the surrounding joints to adjust and compensate for some of the movement that was lost. This can lead to the development of arthritis in these joints. Improving the flexibility or better anatomically mimicking the ankle joint may be an important aspect of ankle arthroplasty treatment.

While total ankle arthroplasty implants may be used to restore the alignment in the ankle joint, replace flexion/extension movement, reduce pain and reduce progression of arthritis in nearby joints, these implants typically last about ten years. As such, a patient may need multiple surgeries due to ankle malfunction. Improving the life of an implant may be an important aspect of ankle arthroplasty treatment.

While a variety of devices and techniques may exist for total ankle replacement, it is believed that no one prior to the inventors have made or used an invention as described herein.

### BRIEF DESCRIPTION OF THE DRAWINGS

While the specification concludes with claims which particularly point out an distinctly claim the invention, it is believed the present invention will be better understood from the following description of certain examples taken in conjunction with the accompanying drawings. In the drawings, like numerals represent like elements throughout the several views.

FIGS. 1(a)-1(f) illustrate an exemplary total ankle replacement assembly.

FIGS. 2(a)-2(f) illustrate a tibial component of the exemplary total ankle replacement assembly of FIG. 1.

FIGS. 3(a)-3(f) illustrate a talar component of the exemplary total ankle replacement assembly of FIG. 1.

FIGS. 4(a)-4(f) illustrate a bearing component of the exemplary total ankle replacement assembly of FIG. 1.

FIGS. 5(a)-5(f) illustrate an exemplary total ankle replacement assembly.

FIGS. 6(a)-6(e) illustrate the tibial component of the exemplary total ankle replacement assembly of FIG. 5.

FIGS. 7(a)-7(f) illustrate the talar component of the exemplary total ankle replacement assembly of FIG. 5.

FIGS. 8(a)-8(f) illustrate the bearing component of the exemplary total ankle replacement assembly of FIG. 5.

FIGS. 9(a)-9(c) illustrate the ring component of the exemplary total ankle replacement assembly of FIG. 5.

FIGS. 10(a)-10(f) illustrate an exemplary total ankle replacement assembly.

FIGS. 11(a)-11(f) illustrates the tibial component of the exemplary total ankle replacement assembly of FIG. 10.

FIGS. 12(a)-12(f) illustrate the talar component of the exemplary total ankle replacement assembly of FIG. 10.

FIGS. 13(a)-13(f) illustrate the bearing component of the exemplary total ankle replacement assembly of FIG. 10.

FIGS. 14(a)-14(f) illustrate an exemplary total ankle replacement assembly.

FIGS. 15(a)-15(f) illustrate the tibial component of the exemplary total ankle replacement assembly of FIG. 14.

FIGS. 16(a)-16(f) illustrate the talar component of the exemplary total ankle replacement assembly of FIG. 14.

FIGS. 17(a)-17(f) illustrate the bearing component of the exemplary total ankle replacement assembly of FIG. 14.

FIGS. 18(a)-18(f) illustrate an exemplary total ankle replacement assembly.

FIGS. 19(a)-19(f) illustrate the tibial component of the exemplary total ankle replacement assembly of FIG. 18.

FIGS. 20(a)-20(f) illustrate the talar component of the exemplary total ankle replacement assembly of FIG. 18.

FIGS. 21(a)-21(f) illustrate the bearing component of the exemplary total ankle replacement assembly of FIG. 18.

FIGS. 22(a)-22(f) illustrate an exemplary total ankle replacement assembly.

FIGS. 23(a)-23(f) illustrate the tibial component of the exemplary total ankle replacement assembly of FIG. 22.

FIGS. 24(a)-24(f) illustrate the talar component of the exemplary total ankle replacement assembly of FIG. 22.

FIGS. 25(a)-25(f) illustrate the bearing component of the exemplary total ankle replacement assembly of FIG. 22.

FIGS. 26(a)-26(f) illustrate an exemplary total ankle replacement assembly.

FIGS. 27(a)-27(f) illustrate the tibial component of the exemplary total ankle replacement assembly of FIG. 26.

FIGS. 28(a)-28(f) illustrate the talar component of the exemplary total ankle replacement assembly of FIG. 26.

FIGS. 29(a)-29(f) illustrate the bearing component of the exemplary total ankle replacement assembly of FIG. 26.

FIGS. 30-31 illustrate a jig assembly of an exemplary total ankle replacement assembly.

FIGS. 32-33 illustrate an upper shaft of the jig assembly of the exemplary total ankle replacement assembly of FIGS. 30-31.

FIGS. 34-35 illustrate a lower shaft of the jig assembly of the exemplary total ankle replacement assembly of FIGS. 30-31.

FIGS. 36-37 illustrate a side bar of the jig assembly of the exemplary total ankle replacement assembly of FIGS. 30-31.

FIGS. 38-39 illustrate a block holder of the jig assembly of the exemplary total ankle replacement assembly of FIGS. 30-31.

FIGS. 40-41 illustrate a cutting block of the jig assembly of the exemplary total ankle replacement assembly of FIGS. 30-31.

FIG. 42 illustrates a short red screw of the jig assembly of the exemplary total ankle replacement assembly of FIGS. 30-31.

FIG. 43 illustrates a long purple screw of the jig assembly of the exemplary total ankle replacement assembly of FIGS. 30-31.

FIG. 44 illustrates a long yellow screw of the jig assembly of the exemplary total ankle replacement assembly of FIGS. 30-31.

The drawings are not intended to be limiting in any way, and it is contemplated that various examples of the invention may be carried out in a variety of ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following detailed description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

Examples described herein include systems operable to totally replace an ankle joint for those persons with an ankle disability, deformity, or are suffering from osteoarthritis or other arthritic conditions. As described in greater detail below, the total ankle replacement assembly generally comprises a tibial component, a talar component, and a bearing component, where the bearing component is positioned between and articulates with the tibial component and talar component to mimic the natural ankle joint movement. The total ankle replacement assembly may further comprise a non-continuous ring. The non-continuous ring may be configured to engage the bearing component and the tibial component. During installation, the total ankle joint replacement assembly may use a jig assembly to aid in installing the replacement ankle joint.

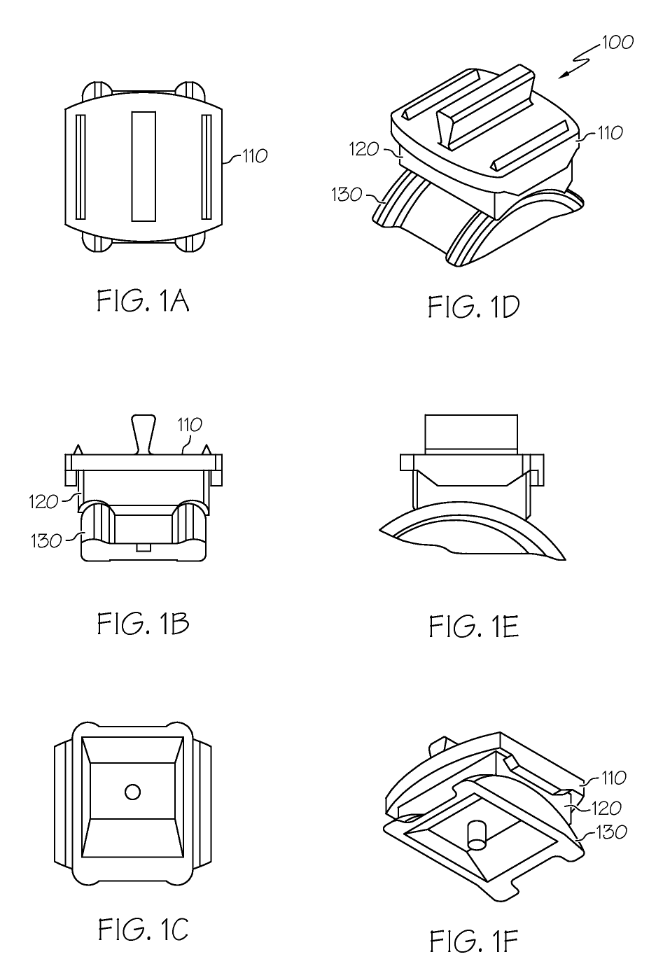

FIGS. 1-4 depict an example of a total ankle joint replacement assembly 100 operable to replace an ankle joint. As shown in FIGS. 1(a)-1(f), the exemplary total ankle joint replacement assembly 100 comprises a tibial component 110, a talar component 130 and a bearing component 120. The bearing component 120 is positioned between and articulates with tibial component 110 and talar component 130 to mimic natural ankle joint movement. The tibial component 110 has an upper surface 111 for contact with the tibia bone of a patient. The talar component 130 has a lower surface 132 for contact with the talus bone of a patient.

The total ankle joint replacement assembly 100 can be designed for bigger ankles or for ankles that may have had previous ankle prosthesis failure. By way of example only, the dimensions of the total assembly 100 can vary as needed to accommodate various ankle sizes and various configurations of an ankle replacement assembly. For example, the width of the total assembly 100 can be about 38 mm. The width of the total assembly 100 can range from about 28 mm to about 48 mm. The width of the total assembly 100 can further range from about 30 mm to about 40 mm. The width of the total assembly 100 can also range from about 36 mm to about 40 mm. In another illustrative example, the length of the total assembly 100 can range from about 29.5 mm to about 50.5 mm. The length of the total assembly 100 can also range from about 34.5 mm to about 45.5 mm. The length of the total assembly 100 can further range from about 38.5 mm to about 41.5 mm. In another illustrative example, the height of the total assembly 100 as measured from the base of the assembly to the upper surface can range from about 7 mm to about 33 mm. The height of the total assembly 100 can also range from about 12 mm to about 28 mm. The height of the total assembly 100 can further range from about 17 mm to about 23 mm. Of course, various other dimensions of the total assembly may be ideal in different patients.

Total assembly 100 can be installed between the tibia bone and talus bone to produce a press-fit connection. Installation may be achieved with or without the use of fixation materials, such as surgical bone cement, e.g., methyl methacrylate, hydroxyapatite, a surgical plate, screws or pins, a combination thereof or any other fixation materials common to one of ordinary skill in the art of joint replacement.

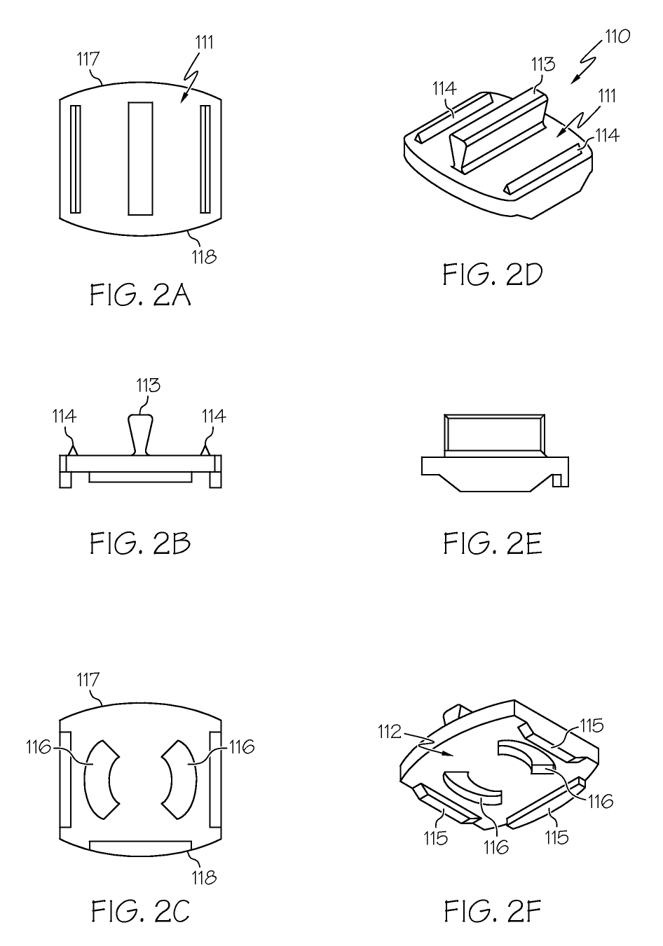

The tibial component 110 of the present example is further depicted in FIGS. 2(a)-2(f). Tibial component 110 comprises an upper surface 111 having projecting members 113, 114 extending from the upper surface 111. The lower surface 112 has projections 115 and recesses 116. At least two edges 117, 118 of the tibial component 110 are shown to be curved.

As referred to herein, tibial components may be made of any metal that can be used to replace a joint, e.g., titanium, titanium alloy, cobalt-chrome, surgical steel, tantalum, etc., or a combination thereof. Of course, the tibial components referred to herein may also be made of various other materials commonly used in the prosthetic arts including, but not limited to, ceramics, plastics, bony ingrowth materials, sintered glass, artificial bone, any other material having excellent wear resistance and is biocompatible, or a combination thereof.

The upper surface 111 and/or projecting members 113, 114 of the tibial component 110 may further be coated with a porous coating, such as a porous mesh, hydroxyapatite, or other biocompatible porous surface, to provide a bony-ingrowth surface. If desired, the upper surface 111 and/or projecting members 113, 114 of the tibial component 110 may be covered with various coatings, such as antimicrobial, antithrombotic, and osteoinductive agents, or a combination thereof. These agents may further be carried in a biodegradable carrier material with which the pores of the upper surface 111 and/or projecting members 113, 114 of the tibial component 110 may be impregnated.

As shown in FIGS. 2(a)-2(f), the projecting members 113, 114 on the upper surface 111 of tibial component 110 are in the shape of three ridges that can run substantially the length of the upper surface 111. Projecting members 113, 114 can help hold the tibial component 110 in place on the tibia and also promote ankle stability during weight bearing. The center projecting member 113 can be tapered at the distal end to provide greater fixation to the tibia bone, and also require greater force necessary for the tibial component 110 to fail and separate from the tibia bone. While the projecting members 113, 114 of the tibial component 110 are depicted in the present example as being rectangular or trapezoidal in shape, various other shapes maybe used according to the teachings herein. The center projecting member 113 can be about five times taller than the two shorter projecting members 114 and can be about three times wider than the shorter projecting members 114. The center projecting member 113 can be situated about equidistance between the shorter projecting members 114. Various dimensions that allow projecting members 113, 114 to better hold the tibial component 110 in place on the tibia will be apparent to those of ordinary skill in the art in view of the teachings herein.

Also depicted are projections 115 on the lower surface 112 of the tibial component 110. The projections 115 extend downwardly from the lower surface 112 of the tibial component 110 and serve to limit rotation of the tibial component 110 when in contact with the bearing component 120. The shape of projections 115 can be trapezoidal, rectangular and/or have various other shapes. By way of example only, projections 115 can extend about 4 mm from the lower surface 112 and can have a thickness of about 2.5 mm. However, projections 115 can extend from the lower surface 112 in a range from about 1 to about 14 mm and can have a thickness from about 1 mm to about 13 mm. The projections 115 can also project from the lower surface 112 in a range from about 3 mm to about 6 mm and can have a thickness from about 1 mm to about 4 mm. Various other dimensions of the projection will be apparent to those of ordinary skill in the art in view of the teachings herein.

Tibial component 110 is also configured to rotate in relation to bearing component 120. FIGS. 2(a)-2(f) depict tibial component 110 having at least two curved recesses 116 disposed in the lower surface 112 that interact with bearing component 120 to provide for rotation in relation to each other. While the present example shows curved recesses, the recesses may be provided in various other shapes, sizes, or configurations as well. By way of example only, the curved recesses 116 can have three radii of curvatures of about 13 mm, about 12.05 mm and about 7.5 mm, although other radii of curvature can be possible. Recesses 116 can be situated equidistance between the projecting members 113, 114 on the upper surface 111 and can have a depth of about 2.5 mm. The depth of recesses 116 can range from about 1 mm to about 13 mm. The depth of the recesses 116 can also range from about 1 mm to about 6 mm.

Tibial component 110 can be installed and secured to a tibia bone. Prior to installation of the tibial component 110, the distal end of the tibia is prepared by cutting away some of the bone. Bone cuts are also made in the tibia bone to form one or more grooves that correspond to one or more projecting members 113, 114 of the tibial component 110. The tibial component 110 is installed by press-fitting one or more projecting members 113, 114 of the tibial component into the one or more corresponding grooves formed in the tibia bone to secure the tibial component 110 with the bone. The tibial component may be further secured in various other ways, including the use of a surgical cement, such as methyl methacrylate, or surgical screws, pins or posts, other fixation materials common to one of ordinary skill in the art of joint replacement, or a combination thereof.

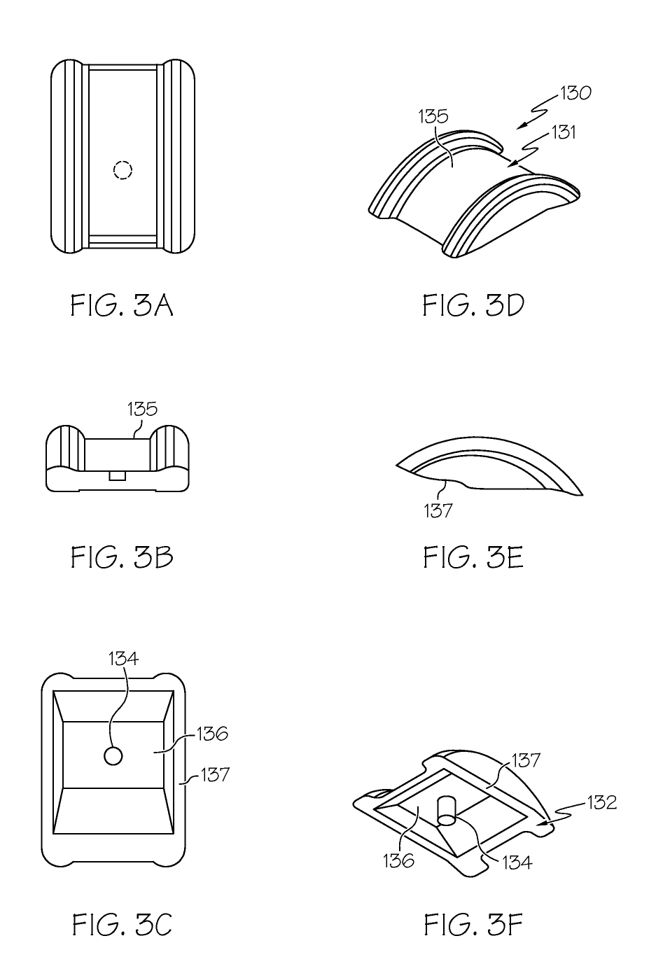

FIGS. 3(a)-3(f) illustrates the talar component 130 of the present example. The talar component comprises a convex upper surface 131, a lower surface 132, and a stem 134 extending from lower surface 132.

As referred to herein, talar components may be made of any metal that can be used to replace a joint, e.g., titanium, titanium alloy, cobalt-chrome, surgical steel, tantalum, etc., or a combination thereof. Of course, the talar components referred to herein may also be made of various other materials commonly used in the prosthetic arts including, but not limited to, ceramics, plastics, bony ingrowth materials, sintered glass, artificial bone, any other material having excellent wear resistance and is biocompatible, or a combination thereof.

The lower surface 132 and/or stem 134 of the talar component 130 may further be coated with a porous coating, such as a porous mesh, hydroxyapatite, or other biocompatible porous surface, to provide a bony-ingrowth surface. If desired, the lower surface 132 and/or stem 134 of the talar component 130 may be covered with various coatings, such as antimicrobial, antithrombotic, and osteoinductive agents, or a combination thereof. These agents may further be carried in a biodegradable carrier material with which the pores of the lower surface 132 and/or stem 134 of the talar component 130 may be impregnated.

The convex upper surface 131 of talar component 130 is curved to mimic the anatomical features of the talus bone. The upper surface 131 can taper along the length of the component 130. The upper surface 131 is also shown having two ribs creating a central groove 135 to produce a surface adapted to slide against the bearing component 120.

Also shown in FIGS. 3(a)-3(f) is the lower surface 132 of the talar component 130 having a large indentation 136 surrounded by thin walls 137 around the perimeter of the lower surface 132. Additionally, talar component 130 is shown having a stem 134 projecting from the indentation 136 in its lower surface 132. Indentation 136 and stem 134 aid in fixation of talar component 130 to the talus bone. The shape of stem 134 can be cylindrical, cross-shaped, or any other suitable shape that can provide bony ingrowth. The shape of indentation 136 can be trapezoidal, semi-spherical, concave, or any other suitable shape.

Talar component 130 can be installed and secured to a talus bone. Prior to installation of the talar component 130, the proximal end of the talus is prepared by cutting away some of the bone so that the talus bone complements the lower surface of the talar component. Bone cuts are also made in the talus bone to form one or more grooves that correspond to the shape of stem 134 of talar component 130. Talar component 130 is installed by press-fitting stem 134 of the talar component into the one or more corresponding grooves formed in the tibia bone to secure the talar component 130 with the bone. The talar component 130 may be further secured in various other ways, including the use of a surgical cement, such as methyl methacrylate, or surgical screws, pins or posts, other fixation materials common to one of ordinary skill in the art of joint replacement, or a combination thereof. While stem 134 of the tibial component 130 is depicted in the present example as being rectangular in shape, various other shapes maybe used according to the teachings herein.

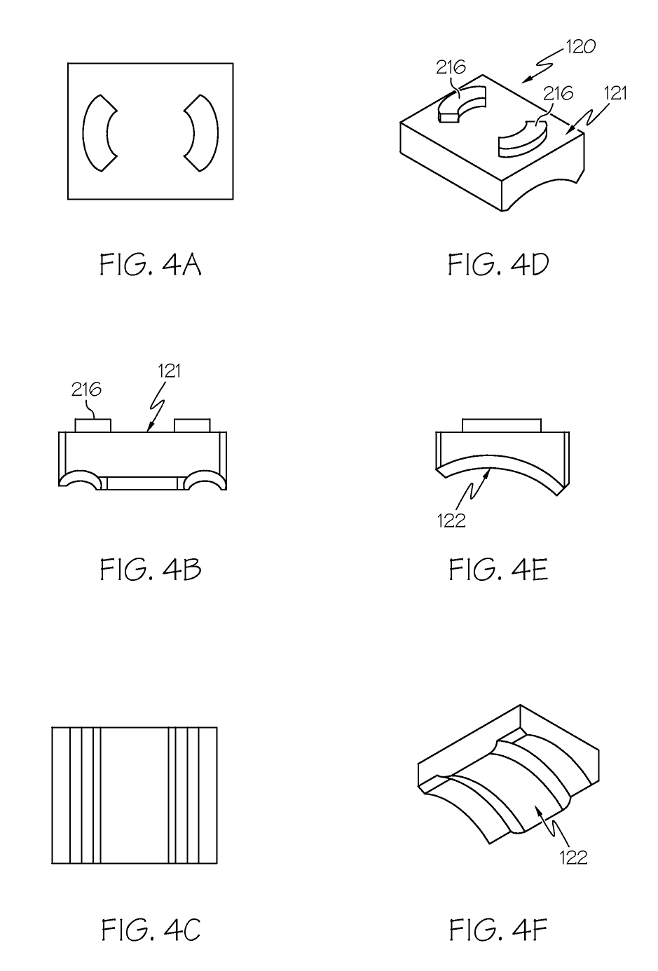

Now turning to FIGS. 4(a)-4(f), the bearing component 120 of the present example is depicted. Bearing component 120 comprises an upper surface, at least one protruding member extending from the upper surface, and a concave lower surface. As referred to herein, bearing components can be comprised of polyethylene or other biocompatible synthetic resins having excellent wear resistance, or a combination thereof.

As shown, bearing component 120 is configured to rotate in relation to tibial component 110. The upper surface 121 has at least two curved protrusions 216 adapted to interact with the at least two curved recesses 116 on tibial component 110 in order to produce rotation in relation to each other. The curved protrusions 216 can have three radii of curvatures of about 12.75 mm, about 11.75 mm and about 8 mm, although other radii of curvature will be apparent to those of ordinary skill in the art in view of the teachings herein. Bearing component 120 can be free and mobile to allow for about ±8° of rotation of the ankle during walking to better mimic natural walking.

The lower surface 122 of the bearing component 120 is adapted to slide against the upper surface 131 of the talar component 130. The lower surface 122 of bearing component 120 has concave curvature that matches and is complementary to the convex curvature of the upper surface 131 of talar component 130. By way of example only, bearing component 120 can have with an outer radius of curvature of 27 mm with a range of about 17 mm to about 37 mm and an inner radius of curvature of about 26 mm with a range of about 16 mm to about 36 mm.

During installation, bearing component 120 is positioned between the tibial component and talar component so that the bearing component articulates with the tibial component and talar component. The bearing component 120 may be seated within the ankle joint between the tibial component and the talar component by hand. The bearing component 120 is inserted when the foot in distracted position. Alternatively, upper surface 121 of bearing component 120 may also be bonded or mechanically attached to the lower surface of the tibial component 110. Bearing component 120 may also be partially locked into the tibial component 110.

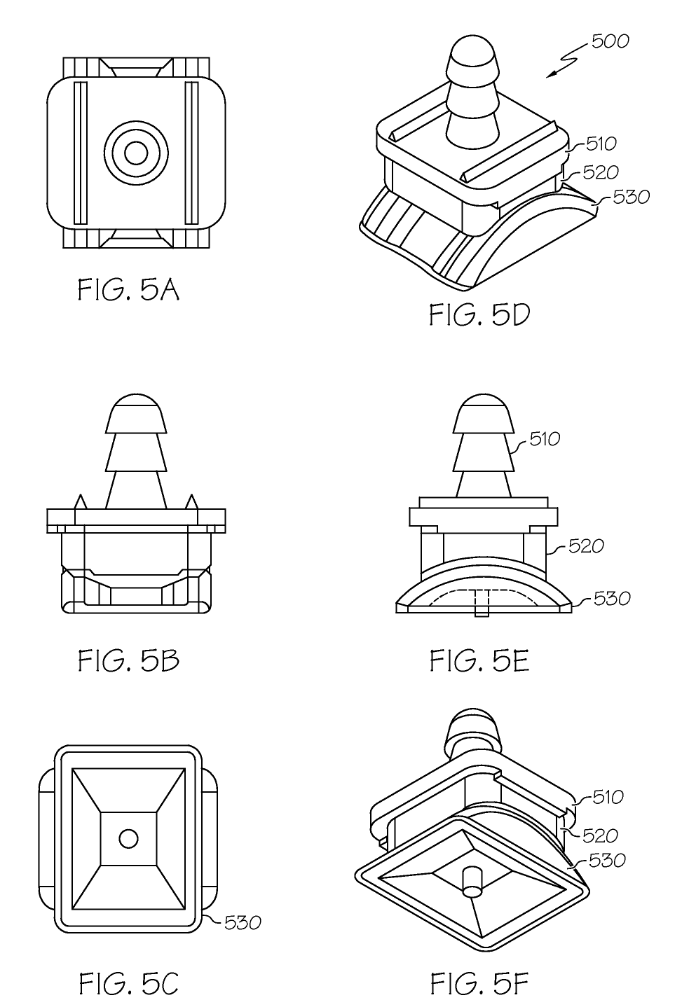

FIGS. 5-8 depict another example of a total ankle joint replacement assembly 500 operable to replace an ankle joint. As shown in FIGS. 5(a)-5(f), the total ankle joint replacement assembly 500 can comprise a tibial component 510, a talar component 530, a bearing component 520 and a non-continuous ring component 540. The bearing component 520 is positioned between and articulates with tibial component 510 and talar component 530 to mimic natural ankle joint movement. Non-continuous ring component 540 is configured to engage the bearing component and tibial component. The tibial component 510 has an upper surface 511 for contact with the tibia bone of a patient. The talar component 530 has a lower surface 532 for contact with the talus bone of a patient. Total assembly 500 of this example is sized, configured and installed in a manner similar to total assembly 100 described above.

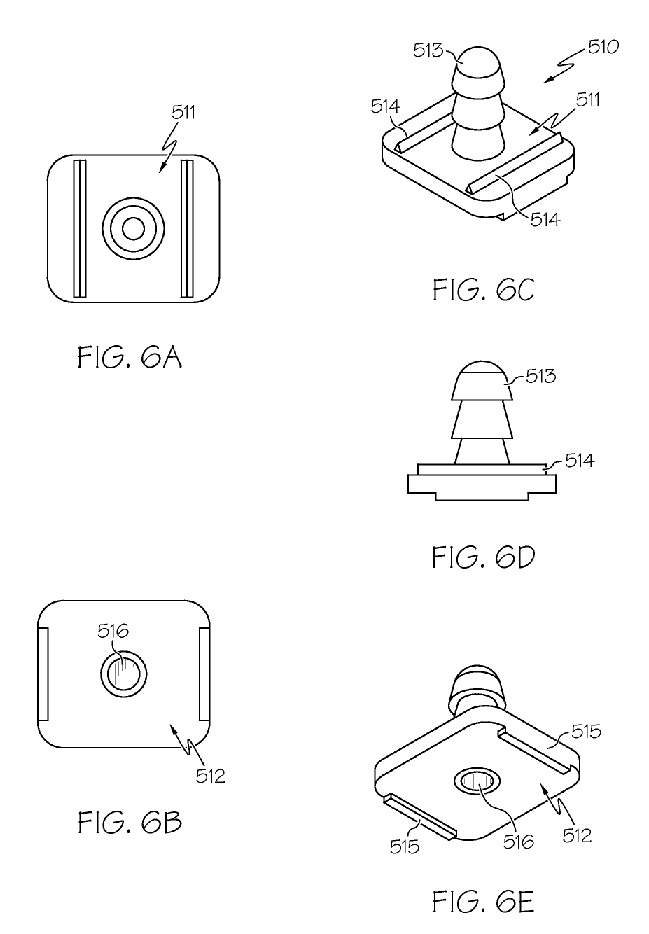

Tibial component 510 is further depicted in FIGS. 6(a)-6(e). Tibial component 510 comprises an upper surface 511 having projecting members 513, 514 extending from the upper surface 511. The lower surface 512 of tibial component 510 has projections 515 and a recess 516.

As shown, projecting members 513, 514 is comprised of a center stem 513 having outwardly extending fins and two ridges 514 positioned on either side of the center stem 513. Projecting members 513, 514 help hold tibial component 510 in place on the tibia, promote ankle stability during weight bearing, and can serve to limit rotation of the tibial component with respect to the tibia bone. Ridges 514 run substantially the length of the tibial component 510. By way of example only, center stem 513 can have a height of about 24 mm, but can range from about 14 mm to about 34 mm. Ridges 514 can have a height of about 2 mm, but can range from about 0.1 mm to about 12 mm, and can have a width in a range of about 1 mm to about 11 mm. Each outwardly extending fin can vary in diameter as you move up center stem 513. Various shapes and dimensions that allow projecting members 513, 514 to better hold tibial component 510 in place on the tibia will be apparent to those of ordinary skill in the art in view of the teachings herein.

Also depicted are projections 515 on the lower surface 512 of the tibial component 510. The projections 515 extend downwardly from the lower surface 512 of the tibial component 510 and serve to limit rotation of tibial component 510 when in contact with the bearing component 520. Projections 515 can be shaped and sized similar to projections 115 as described above. By way of example only, projections 515 can have a thickness in the range of about 1 mm to about 11 mm and a width in the range of about 1 mm to about 11 mm.

Tibial component 510 is also configured to rotate in relation to bearing component 520. FIGS. 6(a)-6(e) depicts tibial component 510 having a dome-shaped recess 516 that interacts with bearing component 520 and non-continuous ring component 540 to provide for rotation in relation to each other. While the present example shows a dome-shaped recess, the recess may be provided in various other shapes, sizes, or configurations as well. Tibial component 510 of this example can be made from the same materials, the upper surface 511 and/or projecting members 513, 514 of tibial component 510 may be further coated with a porous coating, and is installed in a similar manner to tibial component 110 described above.

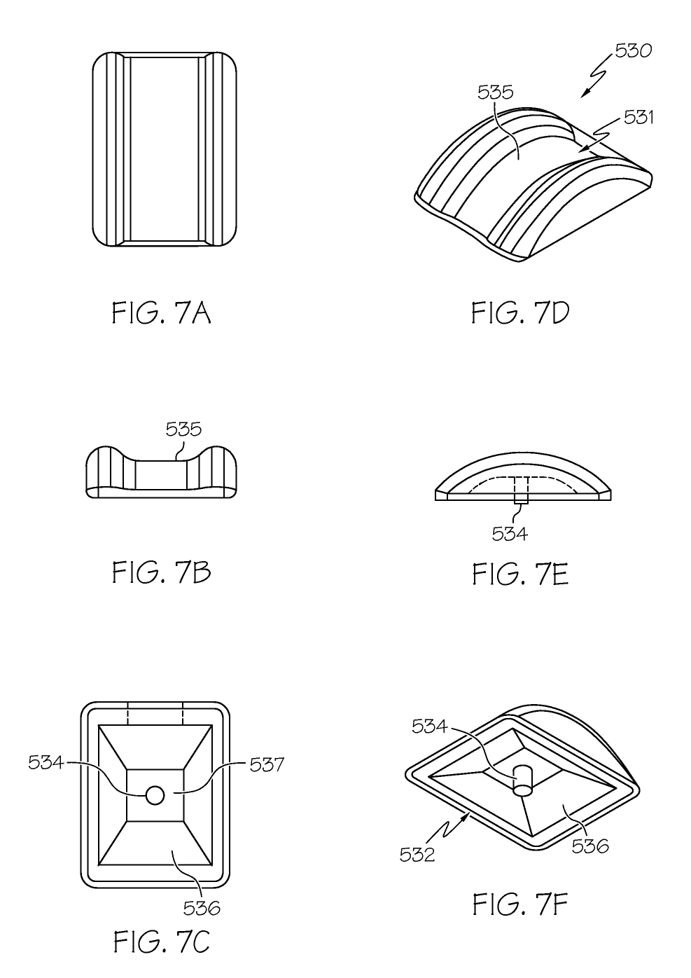

Referring now to FIGS. 7(a)-7(f), talar component 530 of the present example comprises a convex upper surface 531, a lower surface 532, and a stem 534 extending from lower surface 532. Convex upper surface 531 is curved to mimic the anatomical features of the talus bone. Upper surface 531 is shown having two ribs running substantially the length of talar component 530 creating a central groove 535 to produce a surface adapted to slide against bearing component 520.

Also shown is lower surface 532 of talar component 530 having a large indentation 536. The indentation 536 has sloped walls and a lower 537 in the center of indentation 536. Stem 534 is shown protruding from lower 537 of indentation 536. By way of example only, stem 534 can extend about 1 mm from the lower surface 532. Stem 534 aids in fixation of talar component 530 to the talus bone. Talar component 530 of this example can be made from the same materials, the lower surface 532 and/or stem 534 of talar component 530 may be further coated with a porous coating, and is installed in a similar manner to talar component 130 described above.

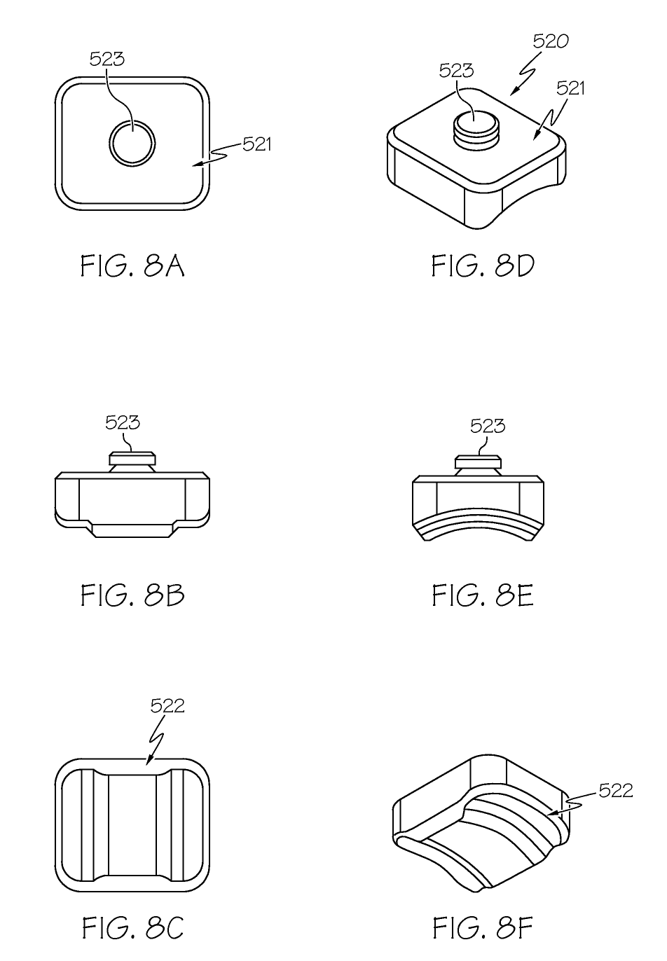

Referring to FIGS. 8(a)-8(f), bearing component 520 of the present example is illustrated. Bearing component 520 comprises an upper surface, at least one protruding member extending from the upper surface, and a concave lower surface.

As shown, bearing component 520 is configured to rotate in relation to tibial component 510. The upper surface 521 has a dome-shaped protruding member 523 adapted to interact with the dome-shaped recess 516 of tibial component 510 in order to produce rotation in relation to each other. Protruding member 523 has a small grove at its base for non-continuous ring component 540 to engage. By way of example only, protruding member 523 can have an outer diameter of about 9 mm, but can range from about 0.1 mm to about 19 mm, and an inner diameter of about 7.25 mm, but can range from about 1 mm to about 18 mm. Bearing component 520 can be free and mobile to allow for up to about ±8° of rotation of the ankle during walking to better mimic natural walking.

The lower surface 522 of the bearing component 520 is adapted to slide against the upper surface 531 of the talar component 530. The lower surface 522 of bearing component 520 has a concave curvature that matches and is complementary to the convex curvature of the upper surface 531 of talar component 530. Bearing component 520 of this example can be made from the same materials and installed in a similar manner to bearing component 120 described above.

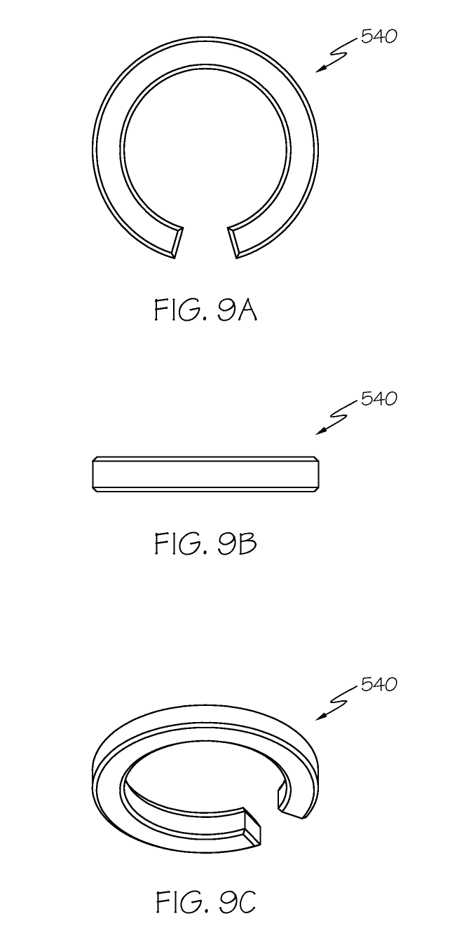

Turning to FIGS. 9(a)-9(c), non-continuous ring component 540 is circular in shape and is configured to engage bearing component 520 and tibial component 510. As referred to herein, non-continuous ring components can be comprised of polyethylene or other biocompatible synthetic resins having excellent wear resistance, or a combination thereof. By way of example only, ring component 540 can have a height of 1.5 mm, but can range from about 0.1 mm to about 12 mm. The inner radius of ring component 540 can be about 3.75 mm, but can range from about 0.1 mm to about 14 mm. The outer radius can be about 5.25 mm, but can range from about 0.1 mm to about 16 mm.

Ring component 540 is configured to be placed in a groove on protruding member 523 of bearing component 520. Protruding member 523 of bearing component 520 may then be inserted into dome-shaped recess 516 of tibial component 510, where ring component 540 expands inside of dome-shaped recess 516. Bearing component 520 is then partially locked into the tibial component 510, but ring component 540 still allows for free rotation of the bearing component 540. The non-continuous ring component 540 can be in a contracted form until inserted inside dome-shaped recess 516 of tibial component 510.

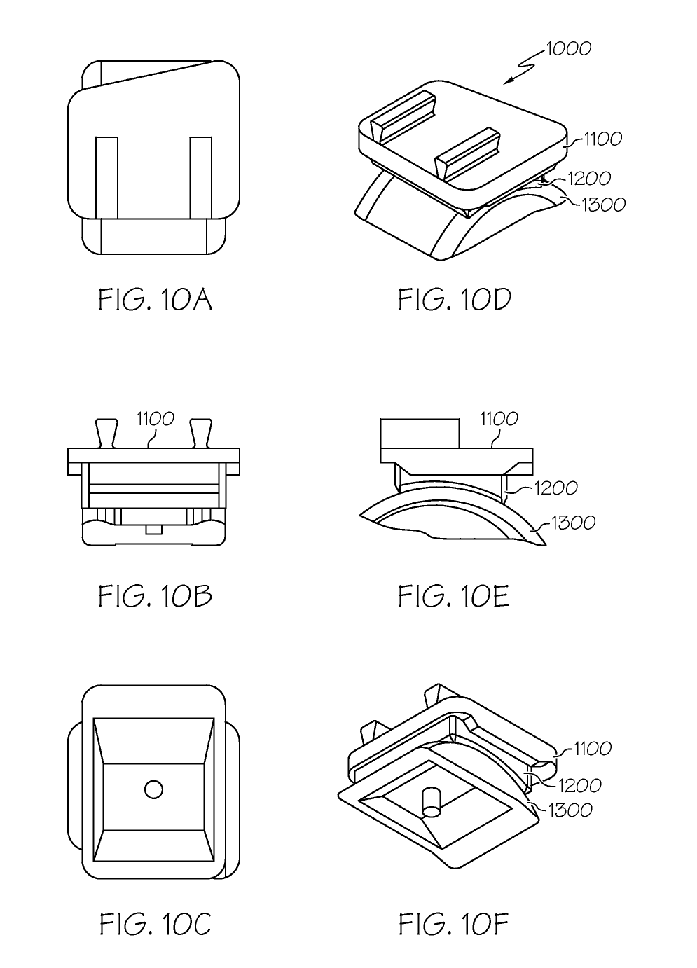

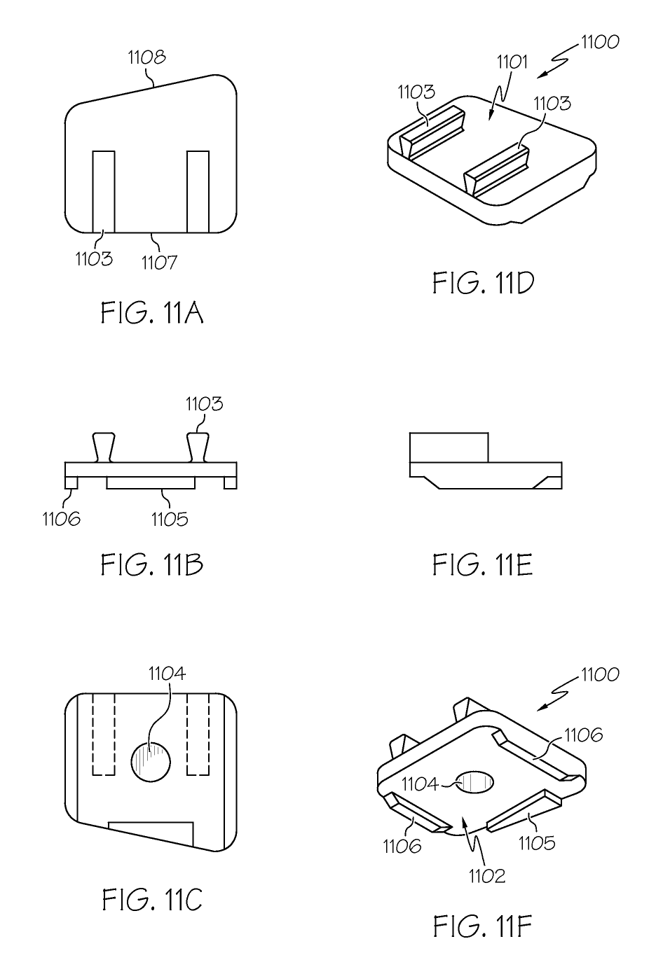

FIGS. 10-13 depict another example of a total ankle joint replacement assembly 1000 operable to replace an ankle joint. As shown in FIGS. 10(a)-10(f), the total ankle joint replacement assembly 1000 can comprise a tibial component 1100, a talar component 1300, and a bearing component 1200. The bearing component 1200 is positioned between and articulates with tibial component 1100 and talar component 1300 to mimic natural ankle joint movement. The tibial component 1100 has an upper surface 1101 for contact with the tibia bone of a patient. The talar component 1300 has a lower surface 1302 for contact with the talus bone of a patient. Total assembly 1000 of this example is sized, configured and installed in a manner similar to total assembly 100 described above.

Turning to FIGS. 11(a)-11(f), the tibial component 1100 of the present example is a trapezoidal in shape and comprises an upper surface 1101, projecting members 1103 extending from the upper surface 1101. The lower surface 1102 of tibial component 1100 has projections 1105, 1106 and a dome-shaped recess 1104.

As shown, projecting members 1103 have two ridges on upper surface 1101 for holding tibial component 1100 in place on the tibia, promoting ankle stability during weight bearing, and serving to limit rotation of the tibial component 1100 with respect to the tibia bone. By way of example only, projecting members 1103 can be about 18 mm long, but can range from about 8 mm to about 28 mm, have a height of about 5.50 mm, but can range from about 0.1 mm to about 16 mm. One end of projecting members 1103 are flush with the straight edge 1107 of tibial component 1100. Various shapes, dimensions, and positions of projecting members 1103 to better hold tibial component 1100 in place on the tibia will be apparent to those of ordinary skill in the art in view of the teachings herein.

Tibial component 1100 is also configured to rotate in relation to bearing component 1200. FIGS. 11(a)-11(f) depict the lower surface 1102 of tibial component 1100 having a dome-shaped recess 1104 that interacts with bearing component 1200 to provide for rotation in relation to each other. While the present example shows a dome-shaped recess, the recess may be provided in various other shapes, sizes, or configurations as well. Tibial component 1100 of this example can be made from the same materials used to make tibial component 110. The upper surface 1101 and/or projecting members 1103 of tibial component 1100 may be further coated with a porous coating as described above related to tibial component 110. Also, tibial component 1100 may be installed in a similar manner as described above with respect to tibial component 110.

Projections 1105, 1106 shown on lower surface 1102 of tibial component 1100 extend downwardly from the edges of three sides on the tibial component 1100, and serve to limit rotation of tibial component 1100 when in contact with bearing component 1200. By way of example only, projection 1106 runs substantially the length of tibial component 1100, and projection 1105 can be wedge-shaped and is positioned in about the center of slanted side 1108 of tibial component 1100. Various shapes, sizes or positions of projections 1105, 1106 will be apparent to one of ordinary skill in the art in view of the teachings herein.

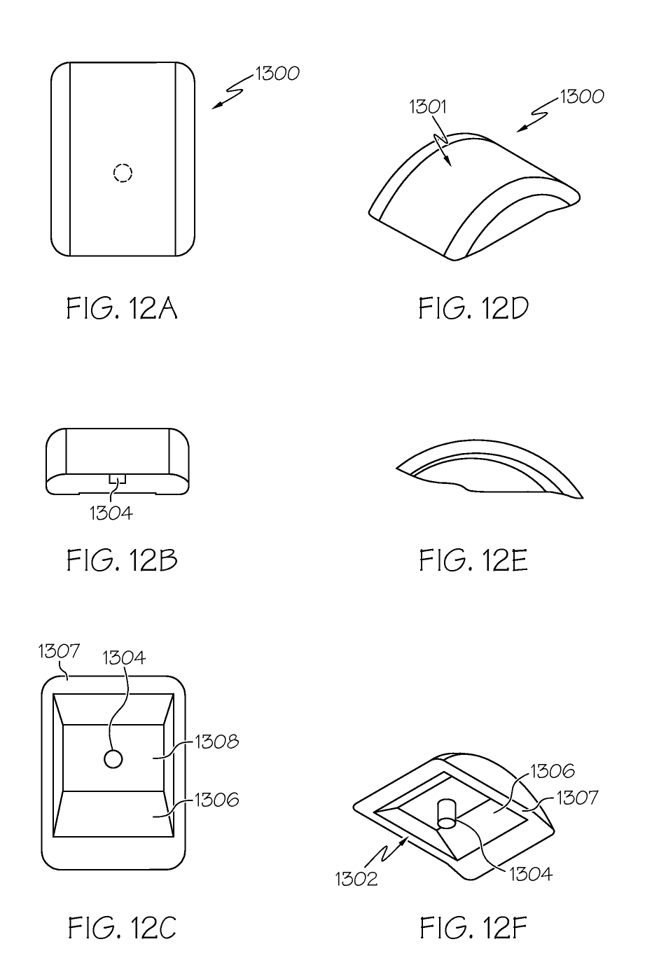

Referring now to FIGS. 12(a)-12(f), the talar component 1300 of the present example comprises a convex upper surface 1301, a lower surface 1302, and a stem 1304 extending from lower surface 1302. Convex upper surface 1301 is curved to mimic the anatomical features of the talus bone and can have a radius of curvature of about 27 mm, but can range from about 17 mm to about 37 mm. Upper surface 1301 of talar component 1300 is adapted to slide against bearing component 1200.

Also shown is lower surface 1302 of talar component 530 having a large indentation 1306 surrounded by a thin walls 1307 around the perimeter of the lower surface 1302. The indentation 1306 has sloped walls and a flat lower 1308 in the center of indentation 1306. Stem 1304 is shown protruding from lower 1308 of indentation 1306. By way of example only, stem 534 can extend about 7 mm from the lower surface 1302. Stem 1304 aids in fixation of talar component 1300 to the talus bone. Talar component 1300 of this example can be made from the same materials used to make talar component 130. The lower surface 1302 and/or stem 1304 of talar component 1300 may be further coated with a porous coating as described above related to talar component 130. Also, talar component 1300 may be installed in a similar manner as described above with respect to talar component 130.

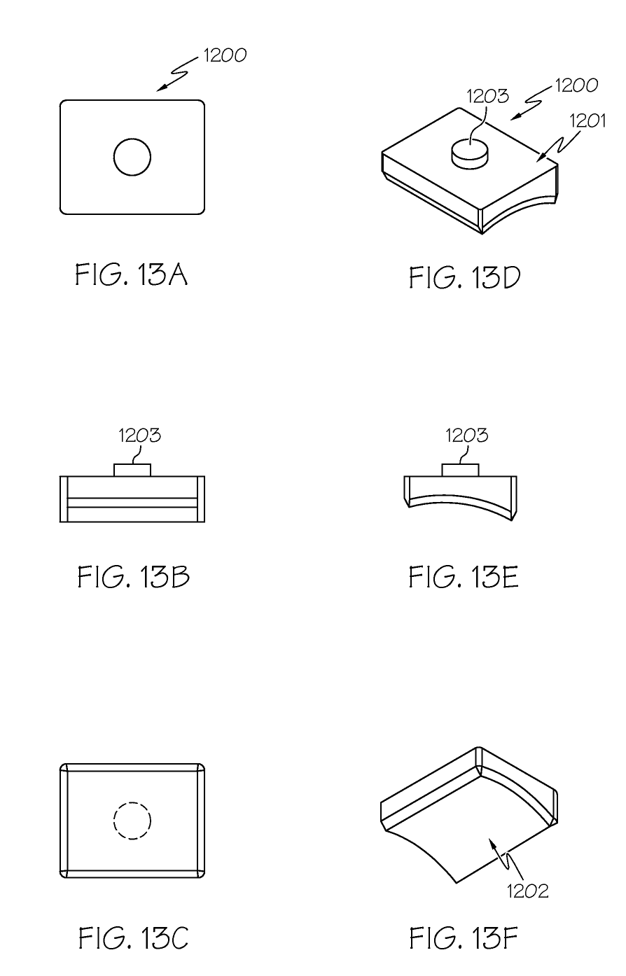

Referring to FIGS. 13(a)-13(f), bearing component 1200 of the present example is illustrated. Bearing component 1200 comprises an upper surface 1201, at least one protruding member 1203 extending from the upper surface 1201, and a concave lower surface 1202.

As shown, bearing component 1200 is configured to rotate in relation to tibial component 1100. The upper surface 1201 has a dome-shaped protruding member 1203 adapted to interact with the dome-shaped recess 1104 of tibial component 1100 in order to produce rotation in relation to each other. By way of example only, protruding member 1203 can have an outer diameter of about 8 mm, but can range from about 0.1 mm to about 18 mm, and an inner diameter of about 6.65 mm, but can range from about 0.1 mm to about 17 mm. Bearing component 1200 can be free and mobile to allow for up to about ±8° of rotation of the ankle during walking to better mimic natural walking.

The lower surface 1202 of the bearing component 1200 is adapted to slide against the upper surface 1301 of the talar component 1300. The lower surface 1202 of bearing component 1200 has a concave curvature that matches and is complementary to the convex curvature of the upper surface 1301 of talar component 1300. Bearing component 1200 of this example can be made from the same materials and installed in a similar manner to bearing component 120 described above.

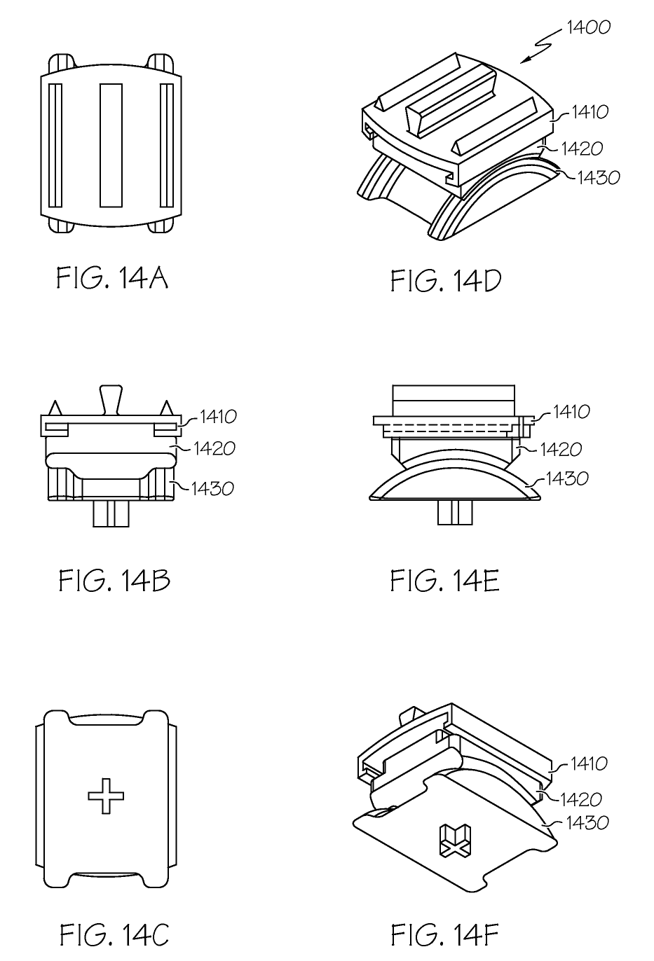

FIGS. 14-17 depict another example of a total ankle joint replacement assembly 1400 operable to replace an ankle joint. As shown in FIGS. 14(a)-14(f), the total ankle joint replacement assembly 1400 can comprise a tibial component 1410, a talar component 1430, and a bearing component 1420. The bearing component 1420 is positioned between and articulates with tibial component 1410 and talar component 1430 to mimic natural ankle joint movement. The tibial component 1410 has an upper surface 1411 for contact with the tibia bone of a patient. The talar component 1430 has a lower surface 1432 for contact with the talus bone of a patient. Total assembly 1400 of this example is sized, configured and installed in a manner similar to total assembly 100 described above.

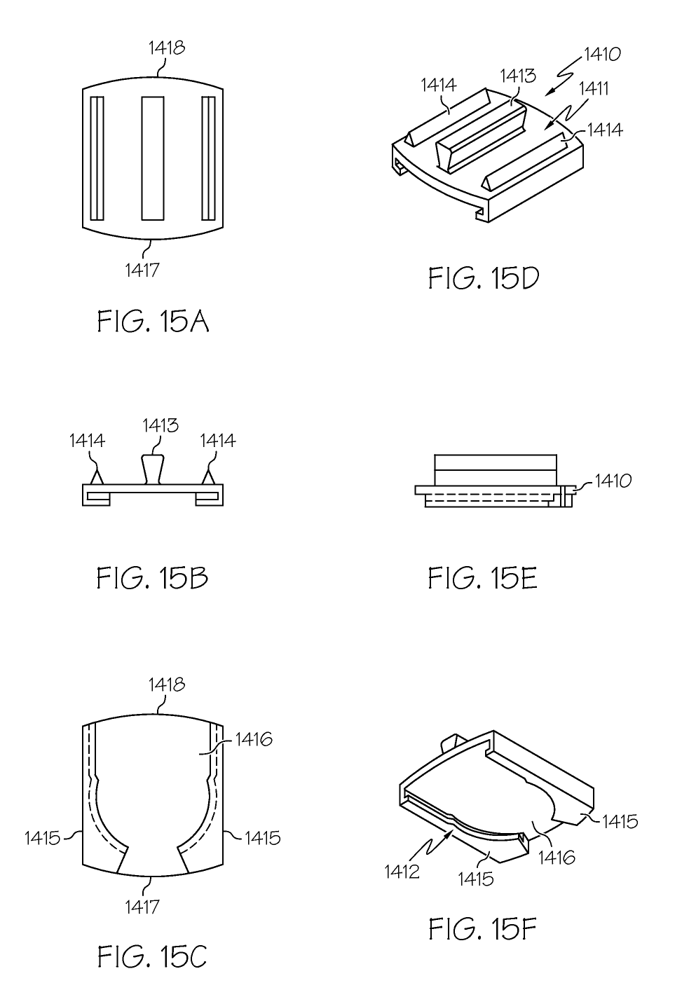

Turning to FIGS. 15(a)-15(f), the tibial component 1410 of the present example is illustrated. Tibial component 1410 comprises an upper surface 1411, projecting members 1413, 1414 extending from the upper surface 1411. The lower surface 1412 of tibial component 1400 has projections 1415 and a recess 1416. At least two edges 1417, 1418 of tibial component 1410 can be curved.

As shown, the projecting members 1413, 1414 on the upper surface 1411 of tibial component 1410 are in the shape of three ridges that can run substantially the length of the upper surface 1411. Projecting members 1413, 1414 can help hold the tibial component 1410 in place on the tibia, promote ankle stability during weight bearing, and can serve to limit rotation of the tibial component 1410 with respect to the tibia bone. The center projecting member 1413 can be tapered at the distal end to provide greater fixation to the tibia bone, and also require greater force necessary for the tibial component 1410 to fail and separate from the tibia bone. While the projecting members 1413, 1414 of the tibial component 1410 are depicted in the present example as being rectangular or trapezoidal in shape, various other shapes maybe used according to the teachings herein. The center projecting member 1413 can be about three to five times taller than the two shorter projecting members 1414 and can be about three times wider than the shorter projecting members 1414. The center projecting member 1413 can be situated about equidistance between the shorter projecting members 1414. Various dimensions that allow projecting members 1413, 1414 to better hold the tibial component 1410 in place on the tibia will be apparent to those of ordinary skill in the art in view of the teachings herein.

Tibial component 1410 is also configured to rotate in relation to bearing component 1420. FIGS. 15(a)-15(f) depict the lower surface 1412 of tibial component 1410 having a recess 1416 that interacts with bearing component 1420 to provide for rotation in relation to each other. The recess 1416 is defined by projections 1415. Projections 1415 shown on lower surface 1412 of tibial component 1410 are curved and extend downwardly from opposite sides of the tibial component 1410, and serve to limit rotation of tibial component 1410 when in contact with bearing component 1420. Tibial component 1410 of this example can be made from the same materials used to make tibial component 110. The upper surface 1411 and/or projecting members 1413, 1414 of tibial component 1410 may be further coated with a porous coating as described above related to tibial component 110. Also, tibial component 1410 may be installed in a similar manner as described above with respect to tibial component 110.

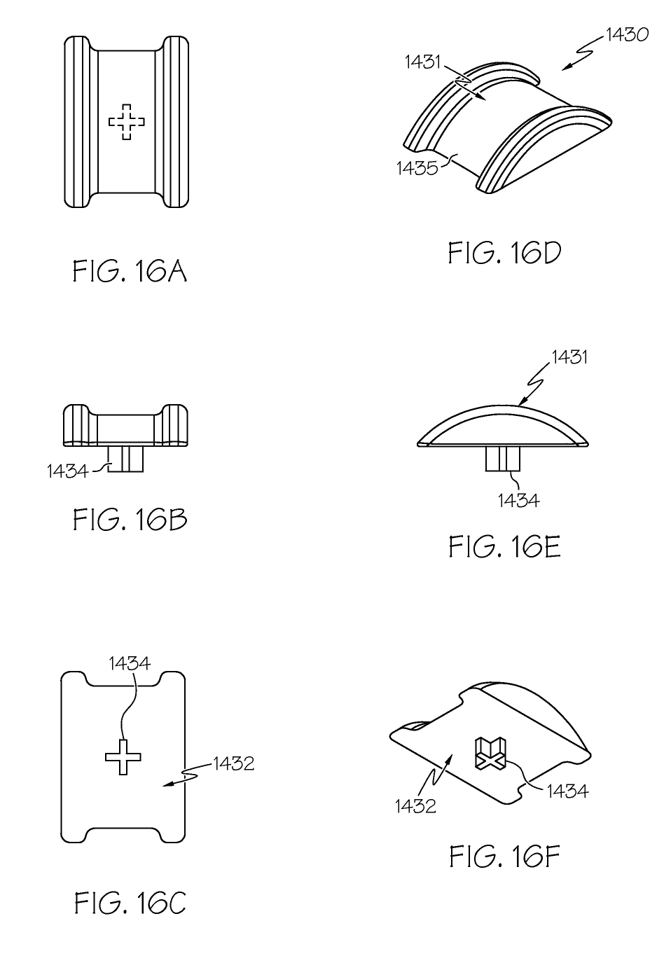

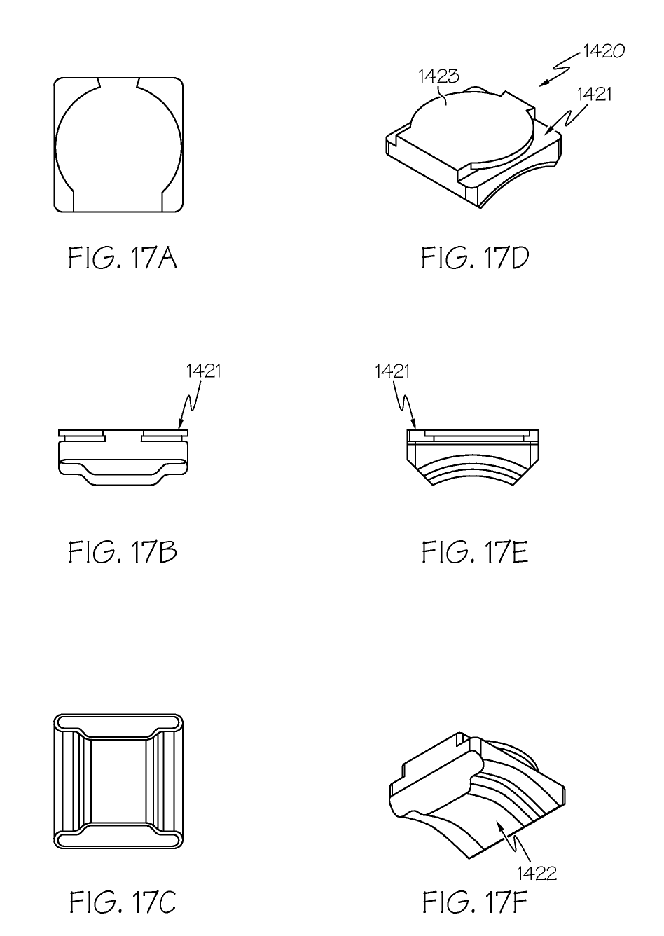

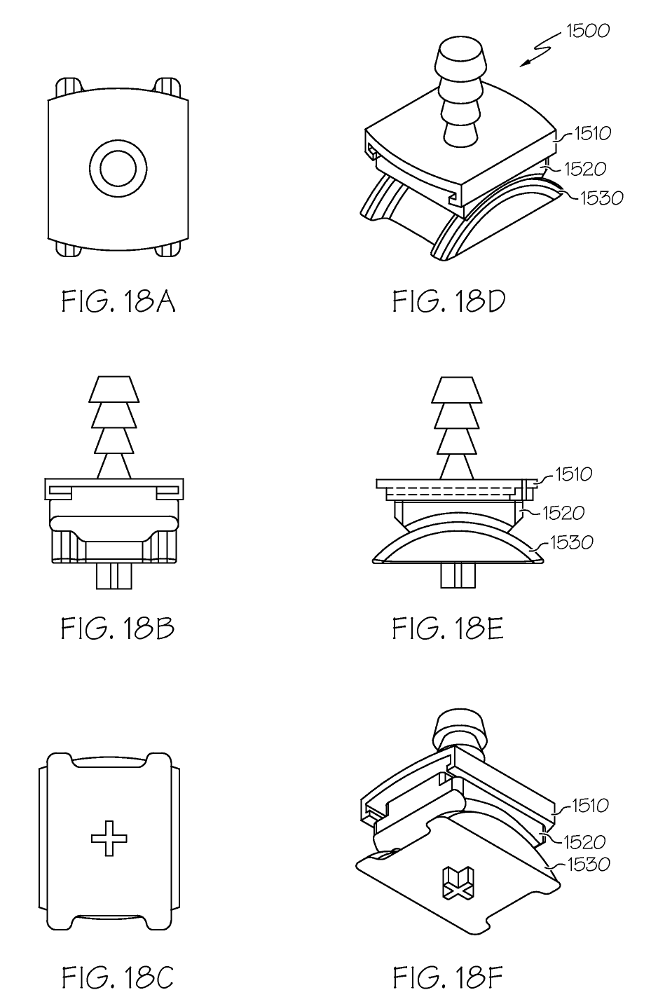

Referring now to FIGS. 16(a)-16(f), talar component 1430 of the present example comprises a convex upper surface 1431, a lower surface 1432, and a stem 1434 extending from lower surface 1432. Convex upper surface 1431 is curved to mimic the anatomical features of the talus bone. Upper surface 1431 is shown having two ribs running substantially the length of talar component 1430 creating a central groove 1435 to produce a surface adapted to slide against bearing component 1420.

Also pictured is lower surface 1432 of talar component 1430 having a flat surface and stem 1434 protruding from the lower surface 1432. Stem 1434 aids in fixation of talar component 1430 to the talus bone. Stem 1434 is shown having a cross-shape, but any other suitable shape can be used in accordance with the teachings herein. Talar component 1430 of this example can be made from the same materials used to make talar component 130. The lower surface 1432 and/or stem 1434 of talar component 1430 may be further coated with a porous coating as described above related to talar component 130. Also, talar component 1430 may be installed in a similar manner as described above with respect to talar component 130.

Referring to FIGS. 17(a)-17(f), bearing component 1420 of the present example is illustrated. Bearing component 1420 comprises an upper surface 1421, at least one protruding member 1423 extending from the upper surface 1421, and a concave lower surface 1422.

As shown, bearing component 1420 is configured to rotate in relation to tibial component 1410. The upper surface 1421 has a protruding member 1423 adapted to interact with the recess 1416 of tibial component 1410 in order to produce rotation in relation to each other. Protruding member 1423 is shaped to match and complement the shape of recess 1416 of tibial component 1410. Bearing component 1420 can be free and mobile to allow for up to about ±8° of rotation of the ankle during walking to better mimic natural walking.

The lower surface 1422 of the bearing component 1420 is adapted to slide against the upper surface 1431 of the talar component 1430. The lower surface 1422 of bearing component 1420 has a concave curvature that matches and is complementary to the convex curvature of the upper surface 1431 of talar component 1430. Bearing component 1420 of this example can be made from the same materials and installed in a similar manner to bearing component 120 described above.

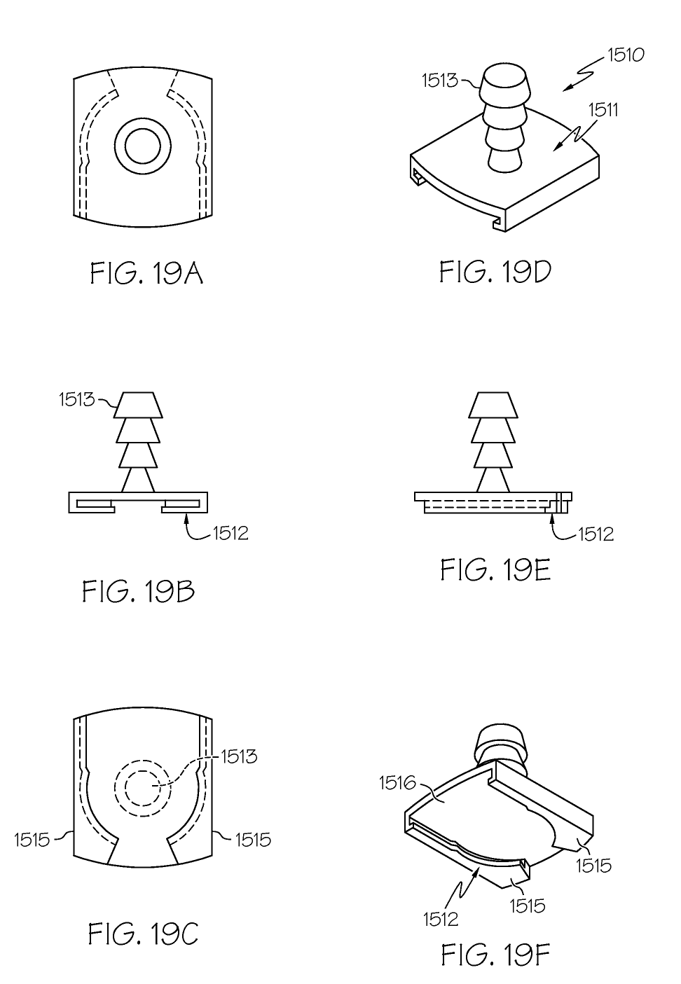

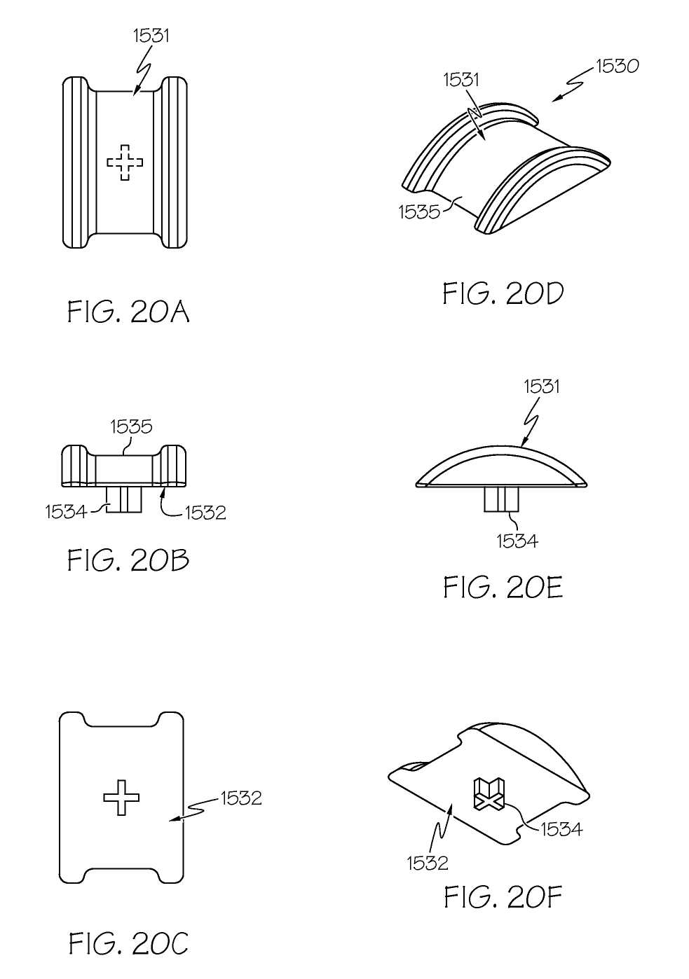

FIGS. 18-21 depict another example of a total ankle joint replacement assembly 1500 operable to replace an ankle joint. As shown in FIGS. 18(a)-18(f), the total ankle joint replacement assembly 1500 can comprise a tibial component 1510, a talar component 1530, and a bearing component 1520. The bearing component 1520 is positioned between and articulates with tibial component 1510 and talar component 1530 to mimic natural ankle joint movement. The tibial component 1510 has an upper surface 1511 for contact with the tibia bone of a patient. The talar component 1530 has a lower surface 1532 for contact with the talus bone of a patient. Total assembly 1500 of this example is sized, configured and installed in a manner similar to total assembly 100 described above.

Tibial component 1510 is further depicted in FIGS. 19(a)-19(f). Tibial component 1510 comprises an upper surface 1511 having projecting member 1513 extending from the upper surface 1511. The lower surface 1512 of tibial component 1510 has projections 1515 and a recess 1516.

As shown, projecting member 1513 is a centrally located stem having outwardly extending fins. Projecting member 1513 helps hold tibial component 1510 in place on the tibia, promote ankle stability during weight bearing, and can serve to limit rotation of the tibial component with respect to the tibia bone. By way of example only, projecting member 513 can have a height of about 25 mm, but can range from about 15 mm to about 35 mm. Each outwardly extending fin can vary in diameter as you move vertically up projecting member 513. Various shapes and dimensions that allow projecting member 1513 to better hold tibial component 1510 in place on the tibia will be apparent to those of ordinary skill in the art in view of the teachings herein.

Tibial component 1510 is also configured to rotate in relation to bearing component 1520. FIGS. 19(a)-19(f) depict the lower surface 1512 of tibial component 1510 having a recess 1516 that interacts with bearing component 1520 to provide for rotation in relation to each other. The recess 1516 is defined by projections 1515. Projections 1515 shown on lower surface 1512 of tibial component 1510 are curved and positioned on opposite sides of the tibial component 1510. Projections 1515 also extend downwardly from lower surface 1512 of tibia component 1510, and serve to limit rotation of tibial component 1510 when in contact with bearing component 1520. Tibial component 1510 of this example can be made from the same materials used to make tibial component 110. The upper surface 1511 and/or projecting member 1513 of tibial component 1510 may be further coated with a porous coating as described above related to tibial component 110. Also, tibial component 1510 may be installed in a similar manner as described above with respect to tibial component 110.

Referring now to FIGS. 20(a)-20(f), talar component 1530 of the present example comprises a convex upper surface 1531, a lower surface 1532, and a stem 1534 extending from lower surface 1532. Convex upper surface 1531 is curved to mimic the anatomical features of the talus bone. Upper surface 1531 is shown having two ribs running substantially the length of talar component 1530 creating a central groove 1535 to produce a surface adapted to slide against bearing component 1520.

Also pictured is lower surface 1532 of talar component 1530 having a flat surface and stem 1534 protruding from the lower surface 1532. Stem 1534 aids in fixation of talar component 1530 to the talus bone. Stem 1534 is shown having a cross-shape, but any other suitable shape can be used in accordance with the teachings herein. Talar component 1530 of this example can be made from the same materials used to make talar component 130. The lower surface 1532 and/or stem 1534 of talar component 1530 may be further coated with a porous coating as described above related to talar component 130. Also, talar component 1530 may be installed in a similar manner as described above with respect to talar component 130.

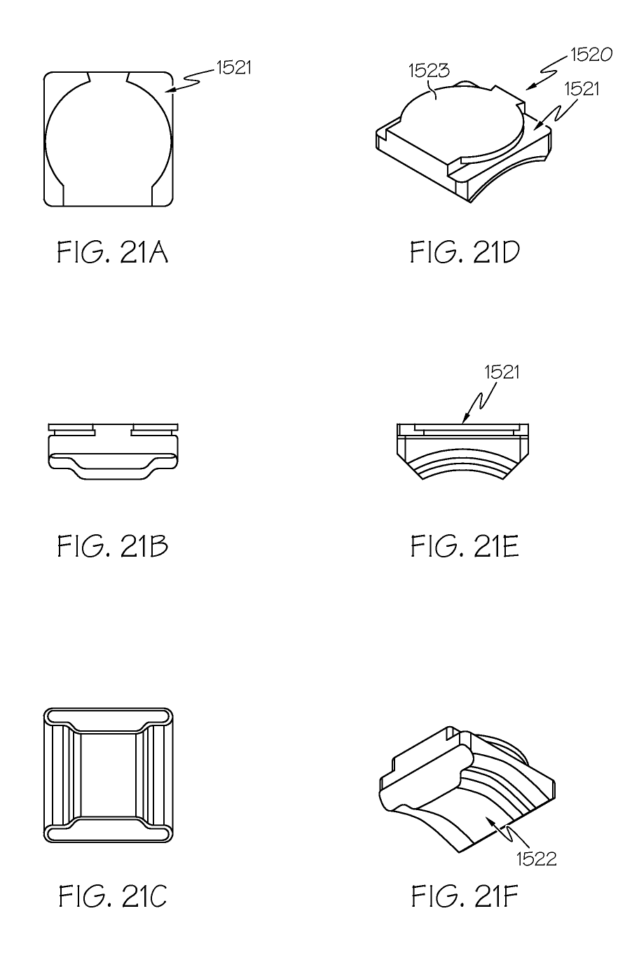

Referring to FIGS. 21(a)-21(f), bearing component 1520 of the present example is illustrated. Bearing component 1520 comprises an upper surface 1521, at least one protruding member 1523 extending from the upper surface 1521, and a concave lower surface 1522.

As shown, bearing component 1520 is configured to rotate in relation to tibial component 1510. The upper surface 1521 has a protruding member 1523 adapted to interact with the recess 1516 of tibial component 1510 in order to produce rotation in relation to each other. Protruding member 1523 is shaped to match and complement the shape of recess 1516 of tibial component 1510. Bearing component 1520 can be free and mobile to have up to about ±8° of rotation of the ankle during walking to better mimic natural walking.

The lower surface 1522 of the bearing component 1520 is adapted to slide against the upper surface 1531 of the talar component 1530. The lower surface 1522 of bearing component 1520 has a concave curvature that matches and is complementary to the convex curvature of the upper surface 1531 of talar component 1530. Bearing component 1520 of this example can be made from the same materials and installed in a similar manner to bearing component 120 described above.

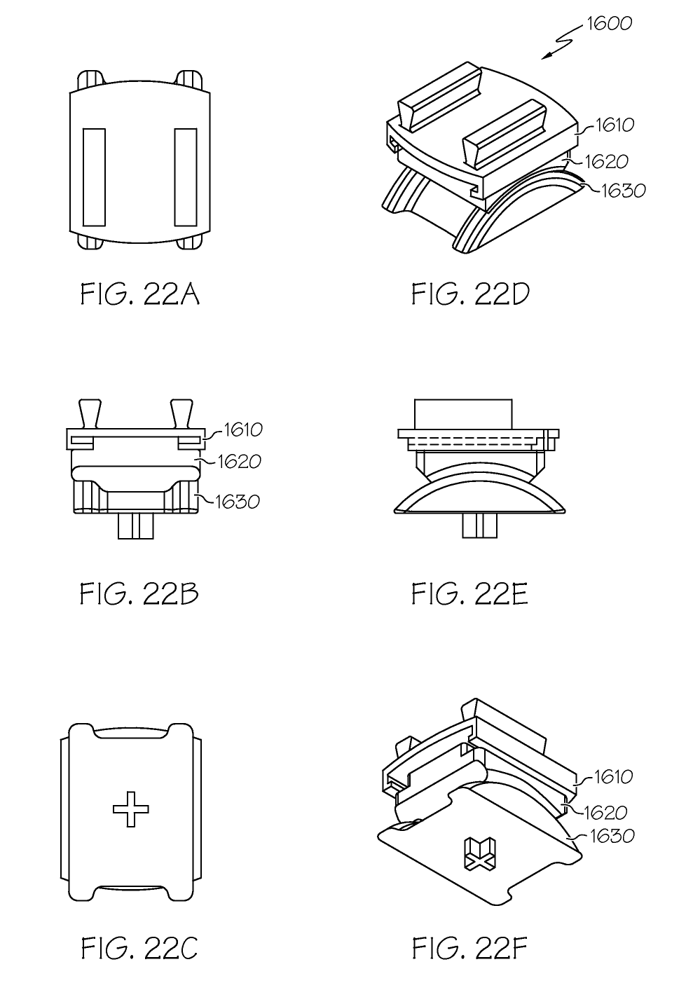

FIGS. 22-25 depict another example of a total ankle joint replacement assembly 1600 operable to replace an ankle joint. As shown in FIGS. 22(a)-22(f), the total ankle joint replacement assembly 1600 can comprise a tibial component 1610, a talar component 1630, and a bearing component 1620. The bearing component 1620 is positioned between and articulates with tibial component 1610 and talar component 1630 to mimic natural ankle joint movement. The tibial component 1610 has an upper surface 1611 for contact with the tibia bone of a patient. The talar component 1630 has a lower surface 1632 for contact with the talus bone of a patient. Total assembly 1600 of this example is sized, configured and installed in a manner similar to total assembly 100 described above.

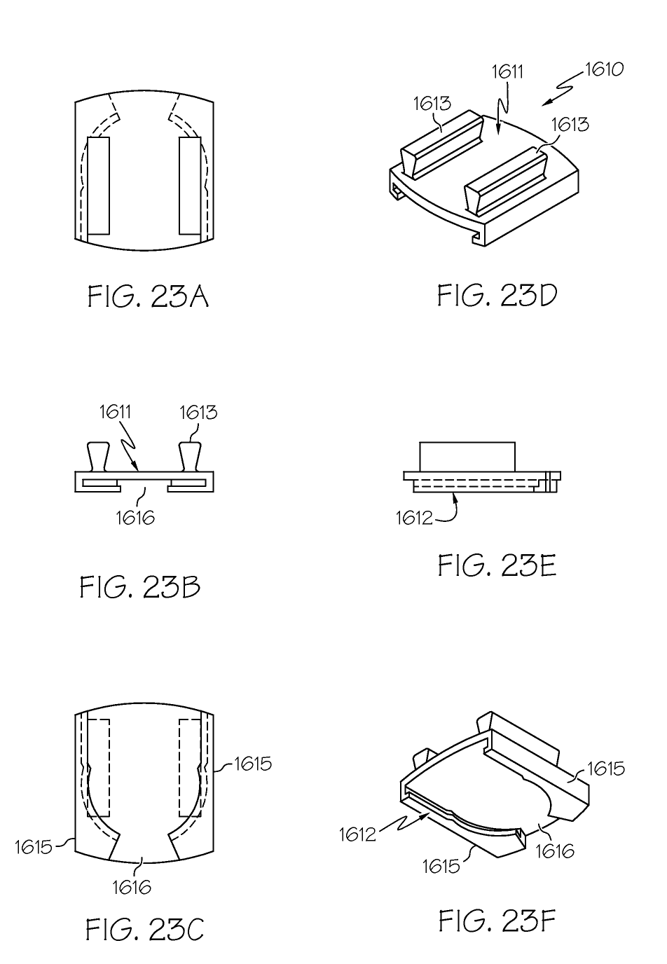

Tibial component 1610 is further depicted in FIGS. 23(a)-23(f). Tibial component 1610 comprises an upper surface 1611 having two projecting members 1613 extending from the upper surface 1611. The lower surface 1612 of tibial component 1610 has projections 1615 and a recess 1616.

As shown, the projecting members 1613 on the upper surface 1611 of tibial component 1610 are in the shape of two ridges that can run substantially the length of the upper surface 1611. Projecting members 1613 can help hold the tibial component 1610 in place on the tibia, promote ankle stability during weight bearing, and can serve to limit rotation of the tibial component 1610 with respect to the tibia bone. The projecting members 1613 can be tapered at the distal end to provide greater fixation to the tibia bone, and also require greater force necessary for the tibial component 1610 to fail and separate from the tibia bone. While the projecting members 1613 of the tibial component 1610 are depicted in the present example as being rectangular or trapezoidal in shape, various other shapes maybe used according to the teachings herein. By way of example only, the height of projecting members 1613 can be about 7 mm, but can range from about 0.1 mm to about 17 mm. Various dimensions that allow projecting members 1613 to better hold the tibial component 1610 in place on the tibia will be apparent to those of ordinary skill in the art in view of the teachings herein.

Tibial component 1610 is also configured to rotate in relation to bearing component 1620. FIGS. 23(a)-23(f) depict the lower surface 1612 of tibial component 1610 having a recess 1616 that interacts with bearing component 1620 to provide for rotation in relation to each other. The recess 1616 is defined by projections 1615. Projections 1615 shown on lower surface 1612 of tibial component 1610 are curved and positioned on opposite sides of the tibial component 1610. Projections 1615 also extend downwardly from lower surface 1612 of tibia component 1610, and serve to limit rotation of tibial component 1610 when in contact with bearing component 1620. Tibial component 1610 of this example can be made from the same materials used to make tibial component 110. The upper surface 1611 and/or projecting members 1613 of tibial component 1610 may be further coated with a porous coating as described above related to tibial component 110. Also, tibial component 1610 may be installed in a similar manner as described above with respect to tibial component 110.

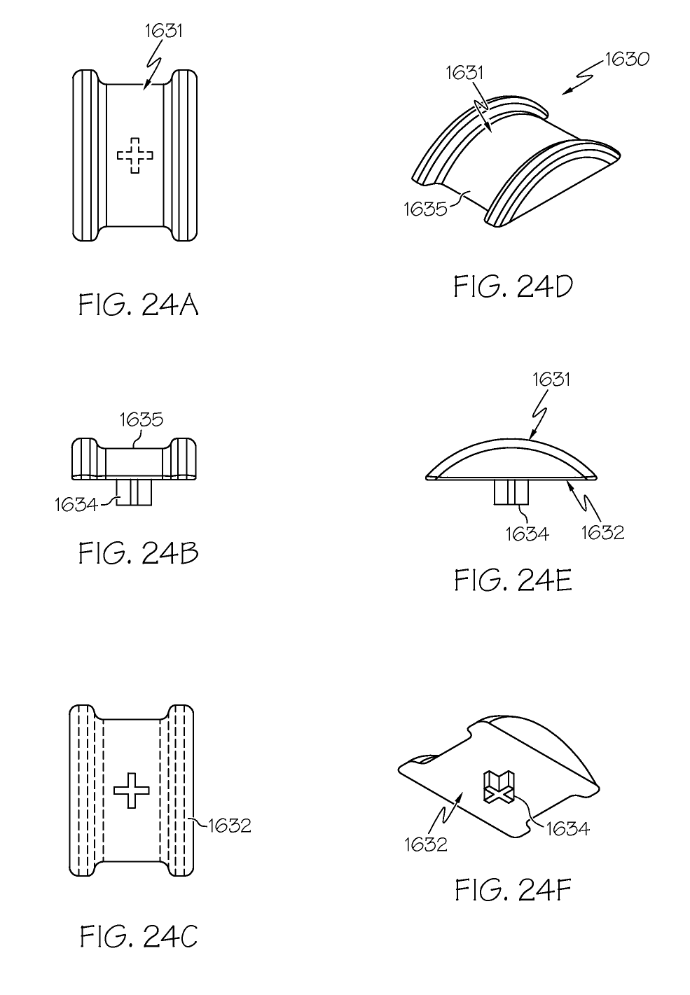

Referring now to FIGS. 24(a)-24(f), talar component 1630 of the present example comprises a convex upper surface 1631, a lower surface 1632, and a stem 1634 extending from lower surface 1632. Convex upper surface 1631 is curved to mimic the anatomical features of the talus bone. Upper surface 1631 is shown having two ribs running substantially the length of talar component 1630 creating a central groove 1635 to produce a surface adapted to slide against bearing component 1620.

Also pictured is lower surface 1632 of talar component 1630 having a flat surface and stem 1634 protruding from the lower surface 1632. Stem 1634 aids in fixation of talar component 1630 to the talus bone. Stem 1634 is shown having a cross-shape, but any other suitable shape can be used in accordance with the teachings herein. Talar component 1630 of this example can be made from the same materials used to make talar component 130. The lower surface 1632 and/or stem 1634 of talar component 1630 may be further coated with a porous coating as described above related to talar component 130. Also, talar component 1630 may be installed in a similar manner as described above with respect to talar component 130.

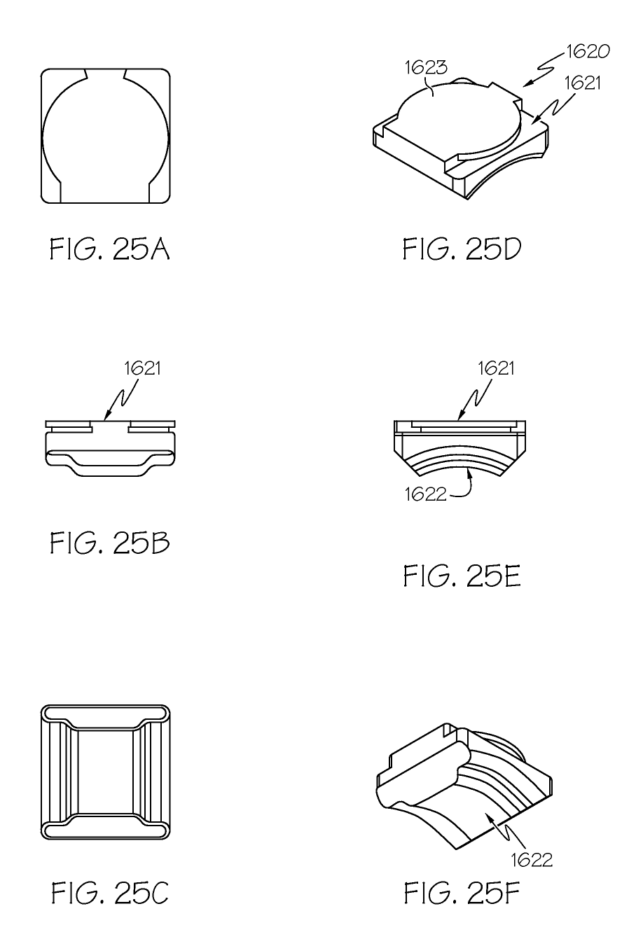

Referring to FIGS. 25(a)-25(f), bearing component 1620 of the present example is illustrated. Bearing component 1620 comprises an upper surface 1621, at least one protruding member 1623 extending from the upper surface 1621, and a concave lower surface 1622.

As shown, bearing component 1620 is configured to rotate in relation to tibial component 1610. The upper surface 1621 has a protruding member 1623 adapted to interact with the recess 1616 of tibial component 1610 in order to produce rotation in relation to each other. Protruding member 1623 is shaped to match and complement the shape of recess 1616 of tibial component 1610. Bearing component 1620 can be free and mobile to have up to about ±8° of rotation of the ankle during walking to better mimic natural walking.

The lower surface 1622 of the bearing component 1620 is adapted to slide against the upper surface 1631 of the talar component 1630. The lower surface 1622 of bearing component 1620 has a concave curvature that matches and is complementary to the convex curvature of the upper surface 1631 of talar component 1630. Bearing component 1620 of this example can be made from the same materials and installed in a similar manner to bearing component 120 described above.

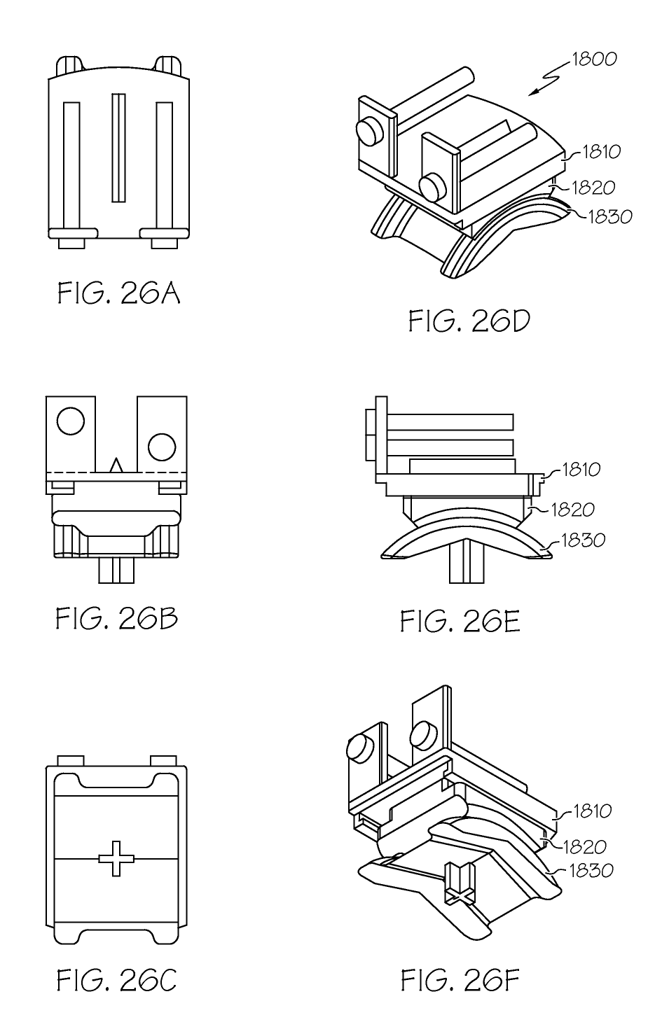

FIGS. 26-29 depict another example of a total ankle joint replacement assembly 1800 operable to replace an ankle joint. As shown in FIGS. 26(a)-26(f), the total ankle joint replacement assembly 1800 can comprise a tibial component 1810, a talar component 1830, and a bearing component 1820. The bearing component 1820 is positioned between and articulates with tibial component 1810 and talar component 1830 to mimic natural ankle joint movement. The tibial component 1810 has an upper surface 1811 for contact with the tibia bone of a patient. The talar component 1830 has a lower surface 1832 for contact with the talus bone of a patient. Total assembly 1800 of this example is sized, configured and installed in a manner similar to total assembly 100 described above.

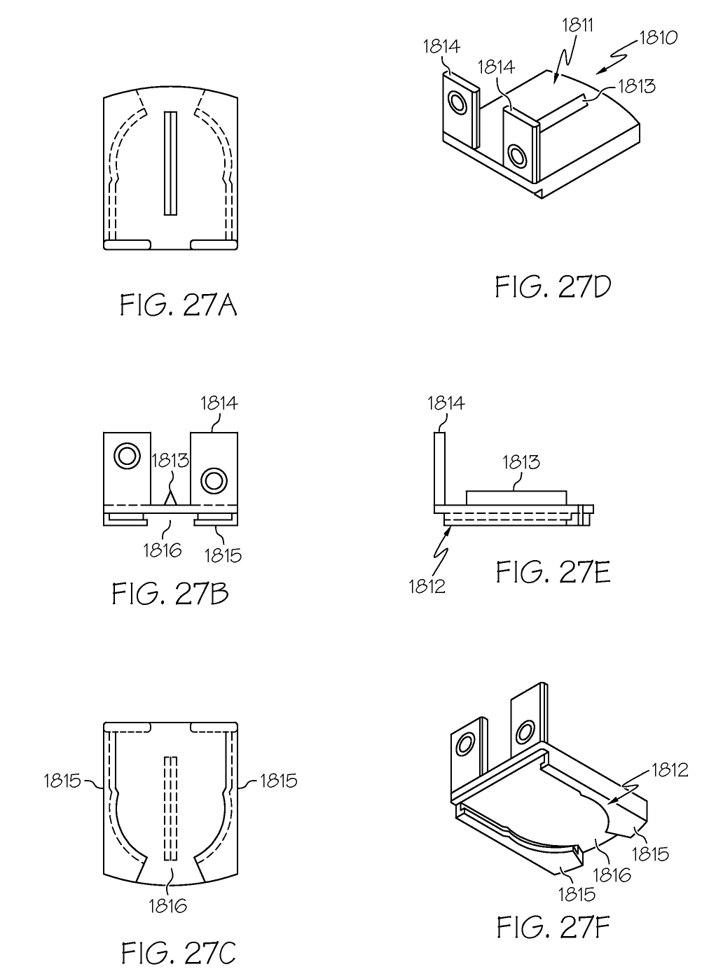

Tibial component 1810 is further depicted in FIGS. 27(a)-27(f). Tibial component 1810 comprises an upper surface 1811 having two projecting members 1813, 1814 extending from the upper surface 1811. The lower surface 1812 of tibial component 1810 has projections 1815 and a recess 1816.

As shown, the projecting member 1813 on the upper surface 1811 of tibial component 1810 is in the shape of a ridge that runs substantially the length of the upper surface 1811. The ridge is shown as being triangular in shape. Of course, various other shapes may be used according to the teachings herein. Projecting members 1814 on the upper surface 1811 of tibial component 1810 are in the shape of two flanges that are transverse to upper surface 1811 and positioned an edge of upper surface 1811. Flanges 1814 each contain an aperture 1817 through which a screw or pin is inserted. Flanges 1814 provide a method of attachment of tibial component 1810 to the tibia bone. Projecting members 1813, 1814 can help hold the tibial component 1810 in place on the tibia, promote ankle stability during weight bearing, and can serve to limit rotation of the tibial component 1810 with respect to the tibia bone. Various dimensions that allow projecting members 1813 to better hold the tibial component 1810 in place on the tibia will be apparent to those of ordinary skill in the art in view of the teachings herein.

Tibial component 1810 is also configured to rotate in relation to bearing component 1820. FIGS. 27(a)-27(f) depict the lower surface 1812 of tibial component 1810 having a recess 1816 that interacts with bearing component 1820 to provide for rotation in relation to each other. The recess 1816 is defined by projections 1815. Projections 1815 shown on lower surface 1812 of tibial component 1810 are curved and positioned on opposite sides of the tibial component 1810. Projections 1815 also extend downwardly from lower surface 1812 of tibia component 1810, and serve to limit rotation of tibial component 1810 when in contact with bearing component 1820. Tibial component 1810 of this example can be made from the same materials used to make tibial component 110. The upper surface 1811 and/or projecting members 1813, 1814 of tibial component 1810 may be further coated with a porous coating as described above related to tibial component 110. Also, tibial component 1810 may be installed in a similar manner as described above with respect to tibial component 110.

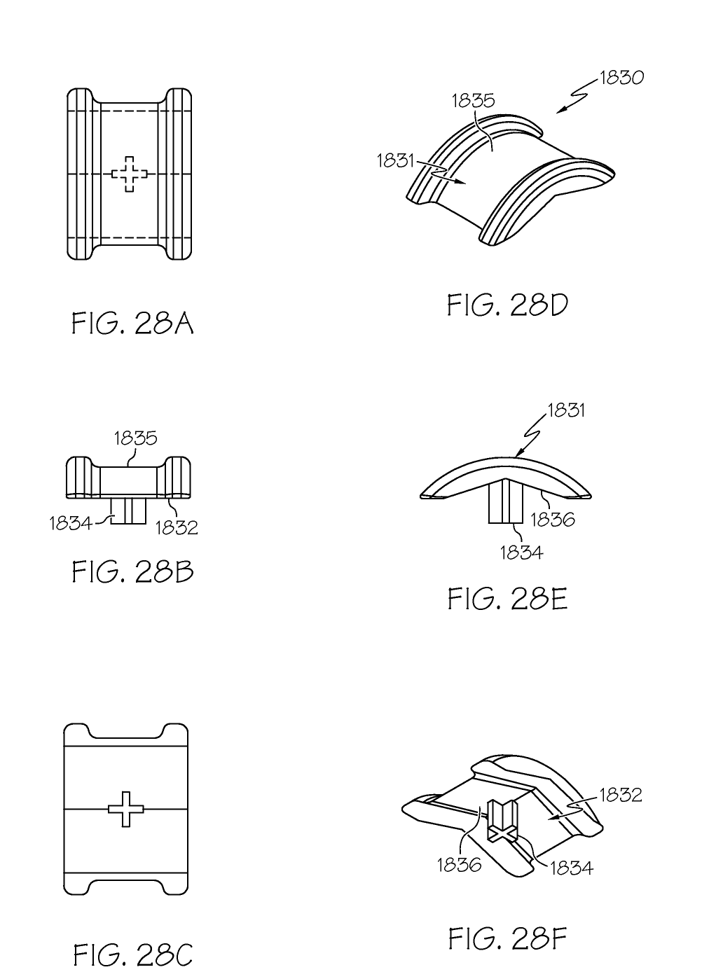

Referring now to FIGS. 28(a)-28(f), talar component 1830 of the present example comprises a convex upper surface 1831, a lower surface 1832, and a stem 1834 extending from lower surface 1832. Convex upper surface 1831 is curved to mimic the anatomical features of the talus bone. Upper surface 1831 is shown having two ribs running substantially the length of talar component 1830 creating a central groove 1835 to produce a surface adapted to slide against bearing component 1820.

Also pictured is lower surface 1832 of talar component 1830 having a wedge-shaped indentation 1836 formed in lower surface 1832 and stem 1834 protruding from lower surface 1832. Stem 1834 aids in fixation of talar component 1830 to the talus bone. Stem 1834 is shown having a cross-shape, but any other suitable shape can be used in accordance with the teachings herein. Talar component 1830 of this example can be made from the same materials used to make talar component 130. The lower surface 1832 and/or stem 1834 of talar component 1830 may be further coated with a porous coating as described above related to talar component 130. Also, talar component 1830 may be installed in a similar manner as described above with respect to talar component 130.

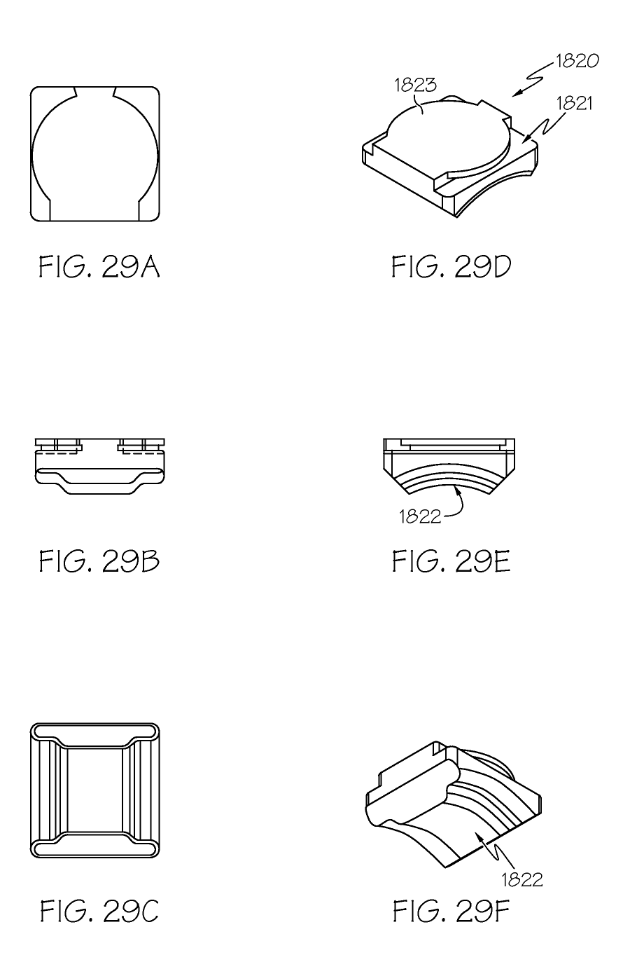

Referring to FIGS. 29(a)-29(f), bearing component 1820 of the present example is illustrated. Bearing component 1820 comprises an upper surface 1821, at least one protruding member 1823 extending from the upper surface 1821, and a concave lower surface 1822.

As shown, bearing component 1820 is configured to rotate in relation to tibial component 1810. The upper surface 1821 has a protruding member 1823 adapted to interact with the recess 1816 of tibial component 1810 in order to produce rotation in relation to each other. Protruding member 1823 is shaped to match and complement the shape of recess 1816 of tibial component 1810. Bearing component 1820 can be free and mobile to have up to about ±8° of rotation of the ankle during walking to better mimic natural walking.

The lower surface 1822 of the bearing component 1820 is adapted to slide against the upper surface 1831 of the talar component 1830. The lower surface 1822 of bearing component 1820 has a concave curvature that matches and is complementary to the convex curvature of the upper surface 1831 of talar component 1830. Bearing component 1820 of this example can be made from the same materials and installed in a similar manner to bearing component 120 described above.

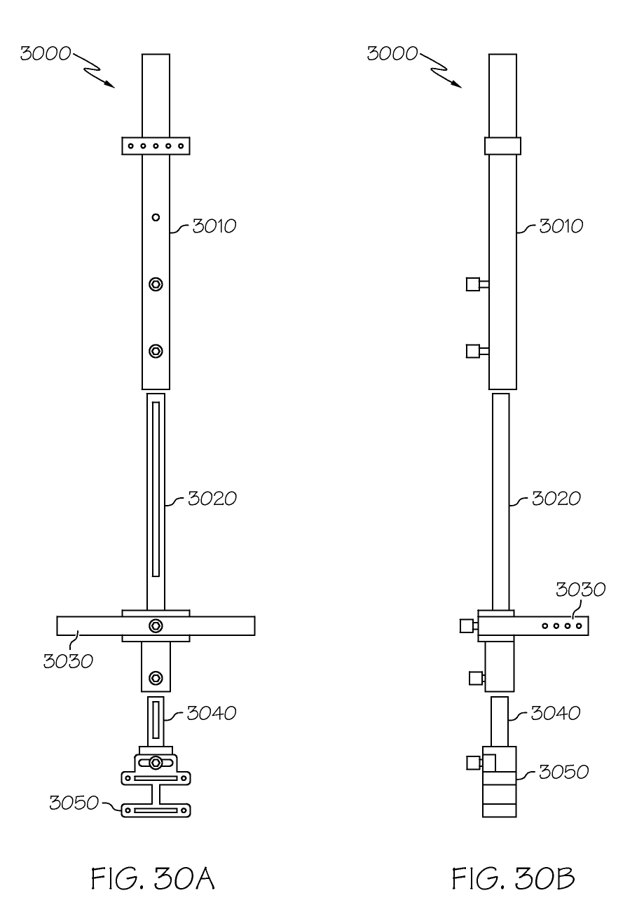

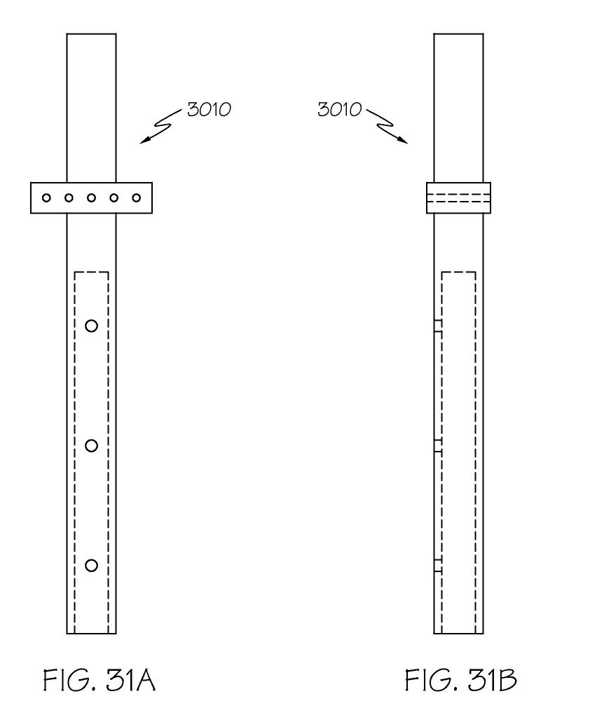

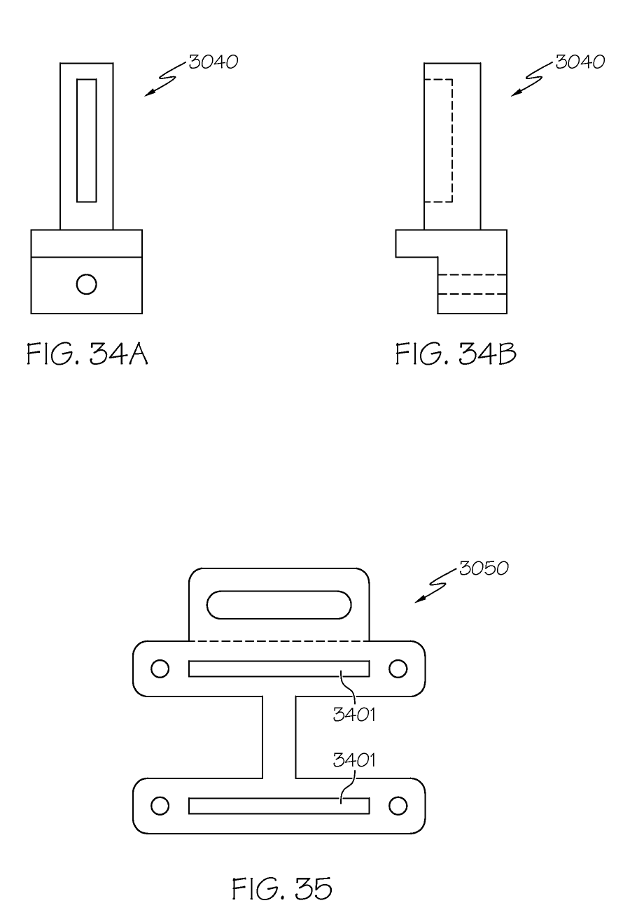

FIGS. 30-41 illustrates an exemplary jig assembly 3000 to be used with the all examples of the total ankle joint replacement assemblies described above when performing an ankle arthroplasty procedure. Jig assembly 3000 can provide precise reproducible bone cuts to the distal end of a tibia bone and proximal end of a talus bone to allow for implantation of the ankle joint assembly. The bone cuts in the tibia and/or talus bones can form a planar surface or surfaces, which may provide greater stability and less stress absorption, and/or decrease the likelihood of the ankle prosthesis loosening.

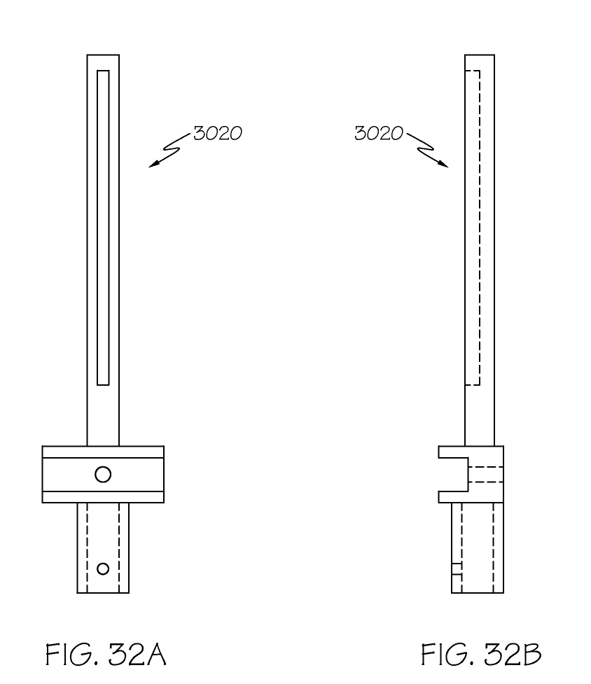

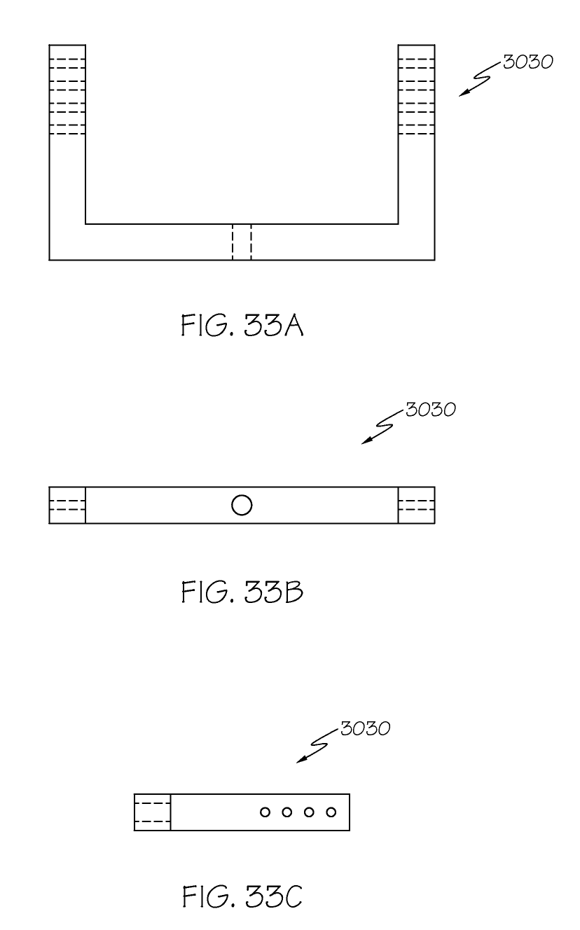

As shown in FIGS. 30(a) and 30(b), the exemplary jig assembly 3000 comprises an upper shaft 3010, which can be further seen in FIGS. 31(a) and 31(b), a lower shaft 3020, which can be further seen in FIGS. 32(a) and 32(b), a side bar 3030, which can be further seen in FIGS. 33(a)-33(c), a block holder 3040, which can be further seen in FIGS. 34(a) and 34(b), and a cutting block 3050, which can be further seen in FIG. 35. The upper shaft 3010 attaches to and is adjustable with respect to the lower shaft 3020. Lower shaft 3020 attaches to and is adjustable with respect to block holder 3040. Side bar 3030 also attaches to lower shaft 3020. Cutting block 3050 attaches to block holder 3040 and is used to provide the reproducible bone cuts through openings 3401. A saw is used through openings 3401 to make the actual bone cuts. Cutting block 3050 can also adjust horizontally as needed.

While several devices and components thereof have been discussed in detail above, it should be understood that the components, features, configurations, and methods of using the devices discussed are not limited to the contexts provided above. In particular, components, features, configurations, and methods of use described in the context of one of the devices may be incorporated into any of the other devices. Furthermore, not limited to the further description provided below, additional and alternative suitable components, features, configurations, and methods of using devices, as well as various ways in which the teachings herein may be combined and interchanged, will be apparent to those of ordinary skill in the art in view of the teachings herein.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A total ankle joint replacement assembly comprising:
a. a tibial component;
b. a talar component; and
c. a bearing component,
wherein the bearing component is positioned between and articulates with the tibial component and talar component to mimic the natural ankle joint movement.

2. The total ankle joint replacement assembly of claim 1, wherein the tibial component has an upper surface, at least one projecting member extending from the upper surface configured to be in contact with a tibia bone, and a lower surface having a recess.

3. The total ankle joint replacement assembly of claim 1, wherein the talar component has a convex upper surface, a lower surface, and a stem extending from the lower surface configured to be in contact with a talus bone.

4. The total ankle joint replacement assembly of claim 1, wherein the bearing component has an upper surface, at least one protruding member extending from the upper surface, and a concave lower surface.

5. The total ankle joint replacement assembly of claim 1, wherein the tibial component and bearing component are configured to rotate in relation to each other.

6. The total ankle joint replacement assembly of claim 1, wherein a lower surface of the bearing component is adapted to slide against an upper surface of the talar component.

7. The total ankle joint replacement assembly of claim 5, wherein the bearing component has a degree of rotation of about +/−8 degrees.

8. The total ankle joint replacement assembly of claim 2, wherein the tibial component further comprises a porous coating on its upper surface.

9. The total ankle joint replacement assembly of claim 3, wherein the talar component further comprises a porous coating on its lower surface.

10. The total ankle joint replacement assembly of claim 1, wherein the assembly further comprises a non-continuous ring component configured to engage the bearing component and the tibial component.

11. A method for replacing an ankle joint having a talus and tibia bone comprising:
a. providing an ankle prosthesis comprising: a tibial component having an upper surface, at least one projecting member on its upper surface, and a lower surface; a talar component having a convex upper surface, a lower surface, and a stem extending from the lower surface; and a bearing component having an upper surface, at least one protruding member extending from the upper surface, and a concave lower surface;
b. installing the tibial component into a tibia bone;
c. installing the talar component into a talus bone;
d. positioning the bearing component between the tibial and talar component so that the bearing component articulates with the tibial component and the talar component.

12. The method of claim 11, wherein the step of installing the tibial component includes press-fitting the at least one projecting member into the tibia bone.

13. The method of claim 11, wherein the step of installing the talar component includes press-fitting the stem into the talus bone.

14. The method of claim 11, wherein the step of positioning the bearing component between the tibial component and talar component produces a press-fit connection.

15. The method of claim 14, wherein the press-fit connection is made without the use of fixation materials.

16. The method of claim 11, wherein the tibial component and bearing component are configured to rotate in relation to each other.

17. The method of claim 11, wherein the lower surface of the bearing component is adapted to slide against an upper surface of the talar component.

18. The method of claim 11, wherein the tibial component has a porous coating on its upper surface.

19. The method of claim 11, wherein the talar component has a porous coating on its lower surface.

20. The method of claim 11, wherein the ankle prosthesis further comprises a non-continuous ring component configured to engage the bearing component and the tibial component.
